(19) United States
(12) Patent Application Publication
(10) Pub. No.: US 2019/0048045 A1
(43) Pub. Date: Feb. 14, 2019

(54) ACTINOBACILLUS PLEUROPNEUMONIAE RECOMBINANT TOXIN PROTEIN AND APPLICATION THEREOF

(71) Applicant: Golden Scorpio International Co., Ltd., Taichung (TW)

(72) Inventors: Ying-Chen YANG, Taipei (TW); Tsun-Yung KUO, I-Lan County (TW)

(21) Appl. No.: 16/171,802

(22) Filed: Oct. 26, 2018

Related U.S. Application Data
(63) Continuation of application No. PCT/CN2016/080469, filed on Apr. 28, 2016.

(51) Int. Cl. C07K 14/285; A61K 39/102; C07K 16/12; G01N 33/569; A61P 31/04; A61K 45/06

(52) U.S. Cl. CPC C07K 14/285; A61K 39/102; C07K 16/1242; A61K 2039/52; A61P 31/04; A61K 45/06; G01N 33/56911

(57) ABSTRACT

The invention relates to a recombinant Actinobacillus pleuropneumoniae toxin, having at least one epitope of Actinobacillus pleuropneumoniae toxin. When there is a plurality of the epitopes, the epitopes can be linked together with linkers. The recombinant Actinobacillus pleuropneumoniae toxin further has at least one unit of the amino acid sequence of complement C3d, and the epitopes and the amino acid sequence of complement C3d can be linked with linkers. The invention also relates to nucleotide sequences encoding the recombinant Actinobacillus pleuropneumoniae toxin and immunogenic compositions containing the recombinant Actinobacillus pleuropneumoniae toxin against porcine pleuropneumonia.

### CROSS-REFERENCE TO RELATED APPLICATION

This application is a continuation application of International Patent Application No. PCT/CN2016/080469, filed on Apr. 28, 2016. The disclosures of the above applications are incorporated herein in their entireties by reference.

Some references, which may include patents, patent applications and various publications, are cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference were individually incorporated by reference.

### FIELD

The present invention relates to preparation and application of recombinant proteins of Actinobacillus pleuropneumoniae, particularly to using recombinant Apx toxins to prevent A. pleuropneumoniae infection in pigs.

### BACKGROUND

Porcine pleuropneumonia is a highly infectious respiratory disease in pigs caused by Actinobacillus pleuropneumoniae (App.). Clinical symptoms of the disease include fever, cough, vomiting, fever, labored breathing, and depression. Infection of the disease can lead to both acute pneumonia with rapid death and chronic infection resulting in asymptomatic carriers. Pigs of all ages are susceptible to porcine pleuropneumonia, in which young pigs less than 6 months have highest morbidity and mortality rates.

A. pleuropneumoniae is a gram-negative rod that has two biotypes. Biotype 1 requires β-nicotinamide adenine dinucleotide (β-NAD), whereas biotype 2 is β-NAD independent. In addition, based on differences in capsular polysaccharides, 15 serotypes of A. pleuropneumoniae have been recognized, and all the 15 serotypes are capable of causing disease. Several virulence factors including capsule, lipopolysaccharides (LPS), outer membrane proteins (OMP) and, most importantly, the Apx toxins are known.

A. pleuropneumoniae RTX (Apx) toxins are members of the repeats in structural toxin (RTX) family. The 15 serotypes of A. pleuropneumoniae produce a total of four Apx toxins, and each serotype is able to produce a maximum of three toxins. The ApxI toxin is produced by serotypes 1, 5, 9, 10, 11, and 14. The ApxII toxin is present in all serotypes but serotypes 10 and 14. The ApxIII toxin is produced by serotypes 2, 3, 4, 6, 8, and 15, and the ApxIV toxin is produced by all serotypes but is expressed solely during infection.

Porcine pleuropneumonia causes great economic losses in swine industry worldwide. Treatment of A. pleuropneumoniae infection involves using antibiotic such as amoxicillin, ampicillin, ceftiofur, enrofloxacin, tiamulin, penicillin, and penicillin/streptomycin. However, due to growing problems of antibiotic resistance and increasing consumer demand for food safety, preventing A. pleuropneumoniae infection by vaccination has become important. Most commercial vaccines against A. pleuropneumoniae infection consist of whole cell bacterins. These vaccines reduce mortality but do not prevent initial infection and the development of the carrier state. Therefore, it is important to develop a more effective vaccine against A. pleuropneumoniae infection.

### SUMMARY

In one aspect, the present invention relates to a recombinant Apx toxin (re-Apx), which is represented by Formula (I):

(A)ₘ-(C3d fragment)ₙ (I)

wherein

each A is an individual epitope of an Apx toxin;

each C3d fragment is an individual unit of the amino acid sequence of complement C3d and is independently selected from the group consisting of SEQ ID NOs: 22, 23, 24, and 25;

m is an integer from 1 to about 30; and

n is an integer from 0 to about 10.

In another aspect, the present invention relates to a nucleotide sequence encoding the recombinant Apx toxin (re-Apx).

In a further aspect, the present invention relates to an immunogenic composition against porcine pleuropneumonia. The immunogenic composition against porcine pleuropneumonia comprises the recombinant Apx toxin (re-Apx) and a pharmaceutically acceptable vehicle.

In another aspect, the present invention relates to a method of protecting an animal against porcine pleuropneumonia. The method comprises administering an effective amount of the immunogenic composition above to the animal to increase immunity against porcine pleuropneumonia in the animal.

In a further aspect, the present invention relates to an anti-Apx toxin antibody. The antibody is prepared or derived from the recombinant Apx toxin (re-Apx) of the present invention.

In yet another aspect, the present invention relates to a test kit for porcine pleuropneumonia. The test kit is used to detect Apx toxin or anti-Apx toxin antibodies in a sample.

The immunogenic composition against porcine pleuropneumonia of the present invention has the following advantages.

The immunogenic composition against porcine pleuropneumonia provided by the present invention comprises a recombinant Apx toxin (re-Apx). The recombinant Apx toxin (re-Apx) comprises at least one epitope of an Apx toxin and partial-length of the amino acid sequence of complement component 3d (C3d). The recombinant Apx toxin (re-Apx) is much shorter than the full-length of an Apx toxin, so the recombinant toxin can be relatively easily expressed in a biological expression system with higher yields and lower costs.

Furthermore, because the recombinant Apx toxin (re-Apx) of the present invention comprises partial-length of the amino acid sequence of C3d, the recombinant protein can increase specific immune response. Experimental data shows that the immunogenic composition against porcine pleuropneumonia comprising the recombinant Apx toxin (re-Apx) of the present invention improves immunity and efficacy against porcine pleuropneumonia in animals.

The present invention is described in more detail in the following illustrative examples. Although the examples may represent only selected embodiments of the invention, it should be understood that the following examples are illustrative and not limiting.

### BRIEF DESCRIPTION OF THE DRAWINGS

The accompanying drawings illustrate one or more embodiments of the invention and together with the written description, serve to explain the principles of the invention. Wherever possible, the same reference numbers are used throughout the drawings to refer to the same or like elements of an embodiment, and wherein:

FIG. 1 shows a sodium dodecyl sulfate polyacrylamide gel electrophoresis (SDS-PAGE) (10%) analysis of purified recombinant Apx toxins (re-Apx) according to Examples 1-4 of the present invention; M: protein markers; lane 1: recombinant ApxI toxin (re-ApxI); lane 2: recombinant ApxII toxin (re-ApxII); lane 3: recombinant ApxIII toxin (re-ApxIII); lane 4: recombinant ApxIV toxin (re-ApxIV).

FIG. 2 shows western blots of recombinant Apx toxins (re-Apx) according to Examples 1-4 of the present invention; M: protein markers; lane 1: recombinant ApxI toxin (re-ApxI); lane 2: recombinant ApxII toxin (re-ApxII); lane 3: recombinant ApxIII toxin (re-ApxIII); lane 4: recombinant ApxIV toxin (re-ApxIV). The primary antibody used in this analysis was alkaline phosphatase-conjugated mouse anti-His Tag antibody (Invitrogen, MA, USA).

FIG. 3 shows results of enzyme-linked immunosorbent assay (ELISA) of antibodies against recombinant ApxII toxin (re-ApxII) according to Example 9 of the present invention; Group 1 is the negative control group (PBS group); Group 2 is the multivalent A. pleuropneumoniae bacterin containing serotypes 1, 2, 5 obtained in Example 6 (App 1, 2, 5 group); Group 3 is the multivalent vaccine containing A. pleuropneumoniae bacterin and recombinant Apx Toxin obtained in Example 6 (App 1, 2, 5+re-ApxI˜III group); Group 4 is a commercial A. pleuropneumoniae bacterin containing serotypes 1, 2, 3, 4, 5, and 7 (App 1, 2, 3, 4, 5, 7 group). ** indicates there is a significant difference between a group and the negative control group (p<0.01); ## indicates there is a significant difference between two groups (p<0.01).

### DETAILED DESCRIPTION

The present invention provides a recombinant Apx toxin (re-Apx). The recombinant toxin has at least one epitope of an Apx toxin to induce animals to produce anti-Apx toxin antibody. The recombinant Apx toxin (re-Apx) may further have at least one unit of partial- or full-length of the amino acid sequence of complement component 3d (C3d) to increase specific immune response. The recombinant Apx toxin (re-Apx) of the present invention may be represented by Formula (I):

(A)ₘ-(C3d fragment)ₙ (I)

wherein

each A is an individual epitope of an Apx toxin;

each C3d fragment is an individual unit of the amino acid sequence of complement C3d;

m is an integer from 1 to about 30; and

n is an integer from 0 to about 10.

In an embodiment, the full-length of the amino acid sequence of the C3d is the full-length of the amino acid sequence of a mouse complement component 3d (mC3d), as shown in SEQ ID NO: 25. In a preferred embodiment, the partial-length of the amino acid sequence of the C3d is the 211^{th} to the 238^{th} amino acids of the mouse C3d (mC3d-p28), which has the sequence of SEQ ID NO: 24. In another embodiment, the full-length of the amino acid sequence of the C3d is the full-length of the amino acid sequence of a pig C3d (pC3d), as shown in SEQ ID NO: 23. In another preferred embodiment, the partial-length of the amino acid sequence of the C3d is the 201⁴ to the 231^{st} amino acids of the pig C3d (pC3d-p31), which has the sequence of SEQ ID NO: 22. The recombinant Apx toxin (re-Apx) of the present invention has 0 to 10 repeated units of the partial- or full-length of the amino acid sequence of C3d, preferably 1 to 10 repeated units, more preferably 4 to 8 repeated units.

In an embodiment, each A is linked to another A with a linker, and each linker is independently selected from Gly-Gly, Gly-Ser, and SEQ ID NOs: 26, 27, 28, 29, 30, 31, 32, 33, 34, 35, and 36.

In an embodiment, each C3d fragment is linked to another C3d fragment with a linker, and each linker is independently selected from Gly-Gly, Gly-Ser, and SEQ ID NOs: 26, 27, 28, 29, 30, 31, 32, 33, 34, 35, and 36.

In an embodiment, an A and a C3d fragment are linked with a linker, and each linker is independently selected from Gly-Gly, Gly-Ser, and SEQ ID NOs: 26, 27, 28, 29, 30, 31, 32, 33, 34, 35, and 36.

In an embodiment, the Apx toxin is ApxI toxin, the recombinant Apx toxin is re-ApxI toxin, and each A is independently selected from the amino acid sequences of SEQ ID NOs: 37, 4, 39, 40, 41, 42, 43, 44, 45, 46, 47, 48, 49, 50, and 51. The number of the epitopes of ApxI toxin is 1 to about 30. The epitopes of ApxI toxin from the N-terminus to the C-terminus of the re-ApxI toxin are not necessarily arranged in ascending or descending order of SEQ ID Numbers. In a preferred embodiment, some of the epitopes of ApxI toxin mentioned above are included in one of five longer epitope fragments of ApxI toxin. The amino acid sequence of each of the five longer epitope fragments of ApxI toxin is SEQ ID NOs: 2, 3, 4, 5, and 6, respectively. In a preferred embodiment, the re-ApxI toxin of the present invention comprises at least two of the longer epitope fragments of ApxI toxin, and the longer epitope fragments of ApxI toxin from the N-terminus to the C-terminus of the re-ApxI toxin are not necessarily arranged in ascending or descending order of SEQ ID Numbers. In a preferred embodiment, the re-ApxI toxin of the present invention comprises the amino acid sequences of SEQ ID NOs: 7 or 8.

In an embodiment, the Apx toxin is ApxII toxin, the recombinant Apx toxin is re-ApxII toxin, and each A is independently selected from the amino acid sequences of SEQ ID NOs: 14, 52, 53, 54, 55, 56, 57, 58, 59, 60, 61, 62, 63, 64, 65, 67, and 68. The number of the epitopes of ApxII toxin is 1 to about 30. The epitopes of ApxII toxin from the N-terminus to the C-terminus of the re-ApxII toxin are not necessarily arranged in ascending or descending order of SEQ ID Numbers. In a preferred embodiment, some of the epitopes of ApxII toxin mentioned above are included in one of five longer epitope fragments of ApxII toxin. The amino acid sequence of each of the five longer epitope fragments of ApxII toxin is SEQ ID NOs: 10, 11, 12, 13, and 14, respectively. In a preferred embodiment, the re-ApxII toxin of the present invention comprises at least two of the longer epitope fragments of ApxII toxin, and the longer epitope fragments of ApxII toxin from the N-terminus to the C-terminus of the re-ApxII toxin are not necessarily arranged in ascending or descending order of SEQ ID Numbers. In a preferred embodiment, the re-ApxII toxin of the present invention comprises the amino acid sequences of SEQ ID NOs: 15 or 16.

In an embodiment, the Apx toxin is ApxIII toxin, the recombinant Apx toxin is re-ApxIII toxin, and each A is independently selected from the amino acid sequences of SEQ ID NOs: 69, 70, 71, 72, 73, 74, 75, 76, 77, 78, 79, 80, 81, 82, 83, 84, 85, 86, 87, and 88. The number of the epitopes of ApxIII toxin is 1 to about 30. The epitopes of ApxIII toxin from the N-terminus to the C-terminus of the re-ApxIII toxin are not necessarily arranged in ascending or descending order of SEQ ID Numbers. In a preferred embodiment, some of the epitopes of ApxIII toxin mentioned above are included in one of two longer epitope fragments of ApxIII toxin. The amino acid sequence of each of the two longer epitope fragments of ApxIII toxin is SEQ ID NOs: 18 and 19, respectively. In a preferred embodiment, the re-ApxIII toxin of the present invention comprises the two longer epitope fragments of ApxIII toxin, and the epitopes of ApxIII toxin from the N-terminus to the C-terminus of the re-ApxIII toxin are not necessarily arranged in ascending or descending order of SEQ ID Numbers. In a preferred embodiment, the re-ApxIII toxin of the present invention comprises the amino acid sequences of SEQ ID NOs: 20 or 21.

In an embodiment, the Apx toxin is ApxIV toxin, the recombinant Apx toxin is re-ApxIV toxin, and each A is independently selected from the amino acid sequences of SEQ ID NOs: 93, 94, 95, 96, 97, 98, 99, 100, 101, 102, 103, 104, 105, 106, 107, 108, 109, 110, 111, 112, 113, 114, 115, 116, and 117. The number of the epitopes of ApxIV toxin is 1 to about 30. The epitopes of ApxIV toxin from the N-terminus to the C-terminus of the re-ApxIV toxin are not necessarily arranged in ascending or descending order of SEQ ID Numbers. In a preferred embodiment, some of the epitopes of ApxIV toxin mentioned above are included in one of three longer epitope fragments of ApxIV toxin. The amino acid sequence of each of the three longer epitope fragments of ApxIV toxin is SEQ ID NOs: 66, 89, and 90, respectively. In a preferred embodiment, the re-ApxIV toxin of the present invention comprises at least two of the longer epitope fragments of ApxIV toxin, and the epitopes of ApxIV toxin from the N-terminus to the C-terminus of the re-ApxIV toxin are not necessarily arranged in ascending or descending order of SEQ ID Numbers. In a preferred embodiment, the re-ApxIV toxin of the present invention comprises the amino acid sequences of SEQ ID NOs: 91 or 92.

In some examples, the recombinant Apx toxin (re-Apx) of the present invention comprises an amino acid sequence having at least about 80%, preferably about 85%, more preferably about 90%, even preferably about 91%, about 92%, about 93%, about 94%, about 95%, about 96%, about 97%, about 98%, or about 99% sequence identity to the amino acid sequence represented by Formula (I).

The invention also provides a polynucleotide sequence encoding the recombinant Apx toxin (re-Apx) of the present invention. The recombinant Apx toxin (re-Apx) comprises at least one epitope of an Apx toxin and 0 to 10 repeated units of the partial- or full-length of the amino acid sequence of C3d. The polynucleotide sequence encoding the recombinant Apx toxin (re-Apx) of the present invention is derived from the amino acid sequence of the recombinant Apx toxin (re-Apx) by replacing each amino acid with a three-nucleotide codon, including every degenerate codon (also called synonymous codons). For example, each serine of the amino acid sequence of the recombinant Apx toxin (re-Apx) can be independently encoded by the codons TCT, TCC, TCA, TCG, AGT, or AGC.

In addition, the present invention provides an immunogenic composition against porcine pleuropneumonia. The immunogenic composition of the present invention comprises the recombinant Apx toxin (re-Apx) of the present invention and a pharmaceutically acceptable vehicle. In an embodiment, the recombinant Apx toxin (re-Apx) is at least one of the re-ApxI toxin, re-ApxII toxin, re-ApxIII toxin, and re-ApxIV toxin. In a preferred embodiment, the immunogenic composition against porcine pleuropneumonia comprises the re-ApxI toxin, re-ApxII toxin, and re-ApxIII toxin and a pharmaceutically acceptable vehicle. In a preferred embodiment, the immunogenic composition against porcine pleuropneumonia comprises the re-ApxI toxin, re-ApxII toxin, re-ApxIII toxin, and re-ApxIV toxin and a pharmaceutically acceptable vehicle.

In an embodiment, the immunogenic composition against porcine pleuropneumonia of the present invention further comprises at least one serotype of A. pleuropneumoniae. The serotype of A. pleuropneumoniae includes, but is not limited to, serotypes 1, 2, 3, 4, 5, 6, 7, 8, 9, 10, 11, 12, 13, 14, and 15 of A. pleuropneumoniae. In a preferred embodiment, the immunogenic composition against porcine pleuropneumonia further comprises serotypes 1, 2, and 5 of A. pleuropneumoniae.

In an embodiment, the immunogenic composition against porcine pleuropneumonia of the present invention further comprises at least one other pathogen antigen. The pathogen antigens include, but are not limited to, antigen of porcine circovirus type 2 (PCV2), antigen of Swine influenza virus (SIV), antigen of porcine reproductive and respiratory syndrome virus (PRRSV), antigen of mycoplasma, antigen of porcine parvovirus (PPV), antigen of erysipelas, antigen of Bordetella bronchiseptica, antigen of Pasteurella multocida, and antigen of pseudorabies (Aujeszky's disease) virus.

The immunogenic composition against porcine pleuropneumonia of the present invention may further comprise at least one pharmaceutically acceptable vehicles including, but not limited to, solvent, emulsifier, suspending agent, decomposer, binding agent, excipient, stabilizing agent, chelating agent, diluent, gelling agent, preservative, lubricant, surfactant, adjuvant, biological carriers, or other suitable vehicles.

The pharmaceutically acceptable vehicle comprises at least one of the following reagents: solvent, emulsifier, suspending agent, decomposer, binding agent, excipient, stabilizing agent, chelating agent, diluent, gelling agent, preservative, lubricant, surfactant, adjuvant, and other suitable vehicles.

The excipient may be pharmaceutically acceptable organic or inorganic carrier substances suitable for parenteral, enteral or intranasal application which do not deleteriously react with the active compounds and are not deleterious to the recipient thereof. Suitable excipients include, but are not limited to, water, salt solutions, vegetable oils, polyethylene glycols, gelatin, lactose, amylose, magnesium stearate, talc, silicic acid, viscous paraffin, fatty acid monoglycerides and diglycerides, petroethral fatty acid esters, hydroxymethyl-cellulose, polyvinylpyrrolidone, etc.

The pharmaceutically acceptable adjuvant includes, but not limited to, aluminum hydroxide, potassium alum, Freund's Complete Adjuvant, Freund's Incomplete Adjuvant, oil adjuvant (such as mineral oil, plant oil, animal oil etc.), aqueous adjuvant, biphasic emulsification adjuvant (such as water-in-oil-in-water emulsion adjuvant), biological adjuvant (such as CpG oligodeoxynucleotide and toxoid), etc. In an embodiment, the adjuvant is aluminum hydroxide.

Furthermore, the invention provides a method of protecting an animal against porcine pleuropneumonia, comprising administering an effective amount of the immunogenic composition of the present invention to the animal to increase immunity against porcine pleuropneumonia in the animal, so that the surviving rate after infection of A. pleuropneumoniae can be improved.

The present invention also provides an anti-Apx toxin antibody prepared or derived from the recombinant Apx toxin (re-Apx) of the present invention. The antibody includes, but is not limited to, monoclonal antibodies, polyclonal antibodies, and genetically engineered antibodies. In an embodiment, the antibody is a polyclonal antibody obtained via injecting an animal with the recombinant Apx toxin (re-Apx).

The present invention further provides a test kit for porcine pleuropneumonia. The test kit is used to detect Apx toxin or anti-Apx toxin antibodies in a test sample. The test kit includes, but is not limited to: (1) an antigen of the recombinant Apx toxin (re-Apx), in one preferred embodiment, the antigen is deposited on an antigen plate, and/or (2) a monoclonal or polyclonal antibody prepared or derived from the recombinant Apx toxin (re-Apx).

The types of the test kit include, but are not limited to, enzyme-linked immunosorbent assay kit (ELISA), microchip kit, immunofluorescent assay (IFA) kit, and other test kits derived from the recombinant Apx toxin (re-Apx). In an embodiment, the test kit comprises at least an antigen plate comprising the recombinant Apx toxin (re-Apx), and the test kit can be used to detect anti-Apx toxin antibodies in a test sample.

As used herein, the singular forms “a”, “an”, and “the” include plural referents unless the context clearly dictates otherwise. Thus, for example, reference to “a component” includes a plurality of such components and equivalents thereof known to those skilled in the art.

As used herein, “around”, “about” or “approximately” shall generally mean within 20 percent, preferably within 10 percent, and more preferably within 5 percent of a given value or range. Numerical quantities given herein are approximate, meaning that the term “around”, “about” or “approximately” can be inferred if not expressly stated.

The meaning of the technical and scientific terms as described herein can be clearly understood by a person of ordinary skill in the art.

The present invention is described in more detail in the following illustrative examples. Although the examples may represent only selected embodiments of the invention, it should be understood that the following examples are illustrative and not limiting.

### EXAMPLE 1

### Construction of Re-ApxI Toxin

Five epitope fragments were selected from the full-length amino acid sequence of Apx I toxin (SEQ ID NO: 1). Sequences of the five epitope fragments are listed below.

Epitope fragment ApxI-1:

(SEQ ID NO: 2)

ELAGITRKGADAKSGK;
Epitope fragment ApxI-2:

(SEQ ID NO: 3)

PAGVGAAA;
Epitope fragment ApxI-3:

(SEQ ID NO: 4)

DILYGSDGTNLFDGGVGNDKIYGG;
Epitope fragment ApxI-4:

(SEQ ID NO: 5)

EHGQVLVGAGGPLAYSNSPNSIPNAF;
Epitope fragment ApxI-5:

(SEQ ID NO: 6)

RAKDELHSVEEIIGSNRKDKFFGSRFTDIFHGAKGDDEIYGNDGHDILYG
DDGNDVIHGGDGNDHLVGGNGNDRLIGGKGNNFLNGGDGDDELLQVFE;

These epitope fragments contain, but are not limited to, the following epitopes:

(SEQ ID NO: 37)

RKGADAKSGK;

(SEQ ID NO: 4)

DILYGSDGTNLFDGGVGNDKIYGG;

(SEQ ID NO: 39)

GGPLAYSNSPNSIPNA;

(SEQ ID NO: 40)

GAGGPLAYSNSPNS;

(SEQ ID NO: 41)

LVGAGGPLAYSNSPNSIPNA;

(SEQ ID NO: 42)

GSNRKD;

(SEQ ID NO: 43)

GAKGDDEIYGNDGHDILYGDDGNDVIHGGDGNDHLVGGNGNDRLIGG;

(SEQ ID NO: 44)

NNFLNGGDGDDEL;

(SEQ ID NO: 45)

GSRFTDIFHGAKGDDEIYGN;

(SEQ ID NO: 46)

DVIHGGDGNDHLVGGNGNDR;

(SEQ ID NO: 47)

IGGKGNNFLNGGDGDDELQV;

(SEQ ID NO: 48)

HGAKGDDEIYGNDG;

(SEQ ID NO: 49)

GGKGNNFLNGGDGD;

(SEQ ID NO: 50)

DGHDILYGDDGNDVIHGGDG;

(SEQ ID NO: 51)

RLIGGKGNNFLNGGDGDDEL.

Epitope fragments ApxI-1, ApxI-2, ApxI-3, ApxI-4, and ApxI-5 were linked from N- to C- terminus, and each epitope fragment was linked to another epitope fragment with at least one linker having the amino acid sequence of SEQ ID NO: 26. Six (6) repeats of pC3d-p31 bioadjuvant (SEQ ID NO: 22) were added to the C-terminus of epitope fragment ApxI-5, in which each pC3d-p31 bioadjuvant was linked to another pC3d-p31 bioadjuvant with at least one linker (SEQ ID NO: 26). The designed recombinant ApxI toxin (re-ApxI) has the amino acid sequence of SEQ ID NO: 7. The amino acid sequence of re-ApxI toxin was synthesized by a peptide synthesizer. Alternatively, a polynucleotide sequence encoding the amino acid sequence of re-ApxI toxin was synthesized and cloned in an expression vector, the expression vector was transfected into a host cell, and then the amino acid sequence of re-ApxI toxin was expressed by the host cell and purified.

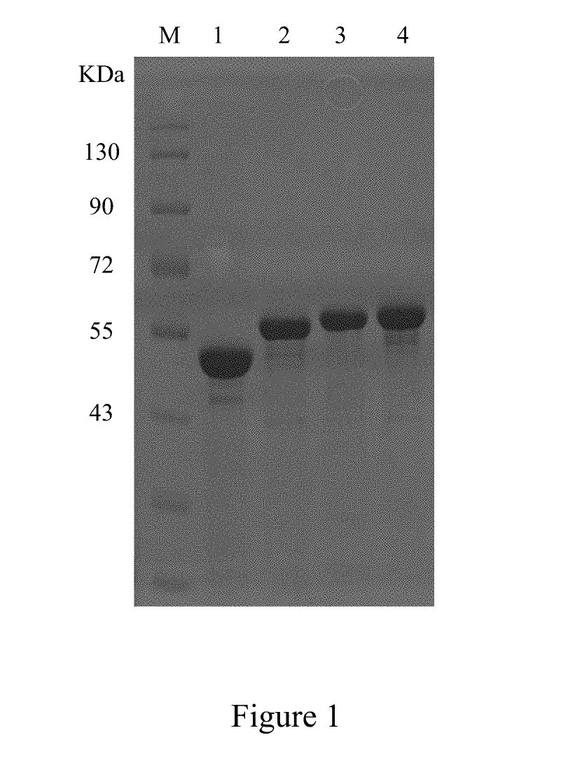

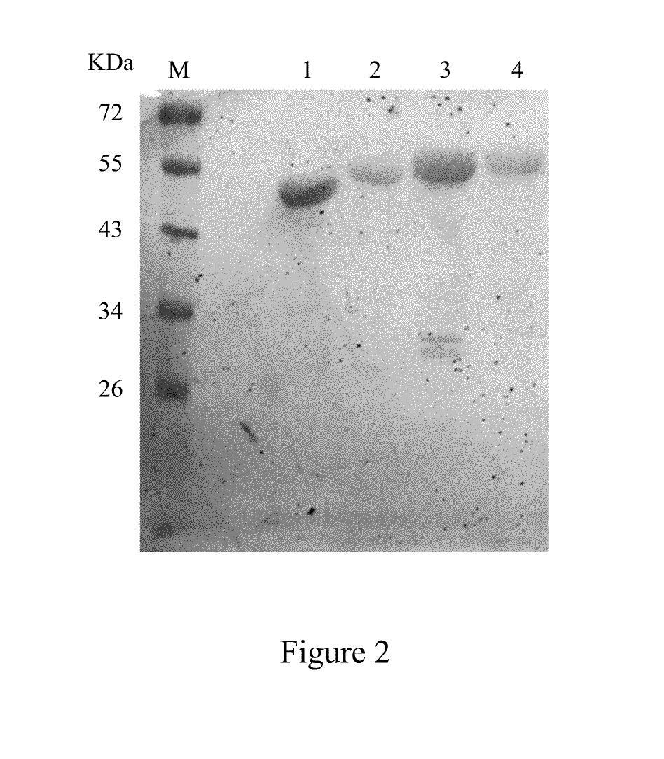

Alternatively, a DNA sequence encoding the amino acid sequence of re-ApxI toxin was constructed by molecular cloning. Then, the polynucleotide sequence was inserted in an expression vector through restriction enzyme sites, and the expression vector was transfected into a host cell. Finally, the amino acid sequence of re-ApxI toxin was expressed by the host cell and purified. In this Example, a DNA sequence encoding the epitope fragments of ApxI toxin and a DNA sequence encoding pC3d-p31 bioadjuvant were linked with a HindIII restriction enzyme site, and the amino acid sequence of re-ApxI toxin obtained by the cloning is SEQ ID NO: 8. The obtained re-ApxI toxin was purified by nickel affinity chromatography and ion exchange chromatography. Then the purified re-ApxI toxin was confirmed by sodium dodecyl sulfate polyacrylamide gel electrophoresis (SDS-PAGE) and western blot, and the results are shown in FIG. 1 (lane 1) and FIG. 2 (lane 2), respectively. The results indicate that the re-ApxI toxin has the expected molecular weight of about 46 kDa.

### EXAMPLE 2

### Construction of Re-ApxII Toxin

Five epitope fragments were selected from the full-length amino acid sequence of ApxII toxin (SEQ ID NO: 9). Sequences of the five epitope fragments are listed below.

Epitope fragment ApxII-1:

(SEQ ID NO: 10)

GNGVDTIDGNDGDDHLFGGAGDDVIDGGNGNNFLVGGTGNDIISGGKDND
IYVHKTGDGNDSITDSGGQDKL;
Epitope fragment ApxII-2:

(SEQ ID NO: 11)

KVGNDYNTSKDRQNV;
Epitope fragment ApxII-3:

(SEQ ID NO: 12)

LTPGEENRERIQEGKNSYITKLHIQRVDSWTVTDGDASSSV;
Epitope fragment ApxII-4:

(SEQ ID NO: 13)

ILYIPQGYDSGQGNGVQDLV;
Epitope fragment ApxII-5:

(SEQ ID NO: 14)

ATHPTNVGNREEKIEYRREDDRFH.

These epitope fragments contain, but are not limited to, the following epitopes:

(SEQ ID NO: 52)

GTGNDIISGGKDNDI;

(SEQ ID NO: 53)

HKTGDGNDSITDSGGQDKL;

(SEQ ID NO: 54)

FGGAGDDVIDGGNGNNFLVG;

(SEQ ID NO: 55)

YVHKTGDGNDSITDSGGQDK;

(SEQ ID NO: 56)

GGAGDDVIDGGNGN;

(SEQ ID NO: 57)

GGTGNDIISGGKDN;

(SEQ ID NO: 58)

KTGDGNDSITDSGG;

(SEQ ID NO: 59)

AGDDVIDGGNGNNFLVGGTG;

(SEQ ID NO: 60)

DIYVHKTGDGNDSITDSGGQ;

(SEQ ID NO: 61)

LTPGEENRERIQEGKNS;

(SEQ ID NO: 62)

WTVTDGDASSSV;

(SEQ ID NO: 63)

TPGEENRERIQEGKNSYITK;

(SEQ ID NO: 64)

LFRTPLLTPGEENR;

(SEQ ID NO: 65)

QGYDSGQGNGVQ;

(SEQ ID NO: 14)

ATHPTNVGNREEKIEYRREDDRF;

(SEQ ID NO: 67)

NREEKIEYRREDDR;

(SEQ ID NO: 68)

PTNVGNREEKIEYRREDDRF.

Epitope fragments ApxII-1, ApxII-2, ApxII-3, ApxII-4, and ApxII-5 were linked from N- to C- terminus, and each epitope fragment was linked to another epitope fragment with at least one linker having the amino acid sequence of SEQ ID NO: 26. Six (6) repeats of pC3d-p31 bioadjuvant (SEQ ID NO: 22) were added to the C-terminus of epitope fragment ApxII-5, in which each pC3d-p31 bioadjuvant was linked to another pC3d-p31 bioadjuvant with at least one linker (SEQ ID NO: 26). The designed recombinant ApxII toxin (re-ApxII) has the amino acid sequence of SEQ ID NO: 15. The amino acid sequence of re-ApxII toxin was synthesized by a peptide synthesizer. Alternatively, a polynucleotide sequence encoding the amino acid sequence of re-ApxII toxin was synthesized and cloned in an expression vector, the expression vector was transfected into a host cell, and then the amino acid sequence of re-ApxII toxin was expressed by the host cell and purified.

Alternatively, a DNA sequence encoding the amino acid sequence of re-ApxII toxin was constructed by molecular cloning. Then, the polynucleotide sequence was inserted in an expression vector through restriction enzyme sites, and the expression vector was transfected into a host cell. Finally, the amino acid sequence of re-ApxII toxin was expressed by the host cell and purified. In this Example, a DNA sequence encoding the epitope fragments of ApxII toxin and a DNA sequence encoding pC3d-p31 bioadjuvant were linked with a HindIII restriction enzyme site, and the amino acid sequence of re-ApxII toxin obtained by the cloning is SEQ ID NO: 16. The obtained re-ApxII toxin was purified by nickel affinity chromatography and ion exchange chromatography. Then the purified re-ApxII toxin was confirmed by sodium dodecyl sulfate polyacrylamide gel electrophoresis (SDS-PAGE) and western blot, and the results are shown in FIG. 1 (lane 2) and FIG. 2 (lane 3), respectively. The results indicate that the re-ApxII toxin has the expected molecular weight of about 47 kDa.

### EXAMPLE 3

### Construction of Re-ApxIII Toxin

Two epitope fragments were selected from the full-length amino acid sequence of Apx III toxin (SEQ ID NO: 17). Sequences of the two epitope fragments are listed below.

Epitope fragment ApxIII-1:

(SEQ ID NO: 18)

ADGDDLLNGNDGDDILYGDKGNDELRGDNGNDQLYGGEGNDKLLGGNGNN
YLSGGDGNDELQVLGNGFNVLRAGKGDDKLYGSSGSDLLDGGEGNDYLEG
GDGSDFYVYRSTSGNHTIYDQGKSSDL;
Epitope fragment ApxIII-2:

(SEQ ID NO: 19)

GELAGITGKGDKLSSGKAYVDYFQEGKLLEKKPDDFSKVVFDPTKGEIDI
SNSQTSTLLKFVTPLLTPGTESRERTQTGK.

These epitope fragments contain, but are not limited to, the following epitopes:

(SEQ ID NO: 69)

GADGDDLLNGNDGDDILYGDKGNDELRGDNGNDQLYGGEGNDKLLGGNGN
NYLSGGDGNDEL;

(SEQ ID NO: 70)

AGKGDDKLYGSSGSDLLDGGEGNDYLEGGDGSD;

(SEQ ID NO: 71)

STSGNHTIYDQGKSSD;

(SEQ ID NO: 72)

GDKGNDELRGDNGNDQLYGG;

(SEQ ID NO: 73)

LLGGNGNNYLSGGDGNDELQ;

(SEQ ID NO: 74)

GDKGNDELRGDNGN;

(SEQ ID NO: 75)

GGNGNNYLSGGDGN;

(SEQ ID NO: 76)

DGDDILYGDKGNDELRGDNG;

(SEQ ID NO: 77)

LLGGNGNNYLSGGDGNDELQ;

(SEQ ID NO: 78)

NGFNVLRAGKGDDKLYGSSG;

(SEQ ID NO: 79)

GITGKGDKLSSGKA;

(SEQ ID NO: 80)

LLEKKPDDF;

(SEQ ID NO: 81)

FDPTKGEIDISNSQT;

(SEQ ID NO: 82)

GITGKGDKLSSGKAYVDYFQ;

(SEQ ID NO: 83)

VFDPTKGEIDISNSQTSTLL;

(SEQ ID NO: 84)

KGDKLSSGKAYVDY;

(SEQ ID NO: 85)

EKKPDDFSKVVFDP;

(SEQ ID NO: 86)

FVTPLLTPGTESRE;

(SEQ ID NO: 87)

ELAGITGKGDKLSSGKAYVD;

(SEQ ID NO: 88)

TLLKFVTPLLTPGTESRERT.

Epitope fragments ApxIII-1 and ApxIII-2 were linked from N- to C- terminus, and each epitope fragment was linked to another epitope fragment with at least one linker having the amino acid sequence of SEQ ID NO: 26. Six (6) repeats of pC3d-p31 bioadjuvant (SEQ ID NO: 22) were added to the C-terminus of epitope fragment ApxIII-2, in which each pC3d-p31 bioadjuvant was linked to another pC3d-p31 bioadjuvant with at least one linker (SEQ ID NO: 26). The designed recombinant ApxIII toxin (re-ApxIII) has the amino acid sequence of SEQ ID NO: 20. The amino acid sequence of re-ApxIII toxin was synthesized by a peptide synthesizer. Alternatively, a polynucleotide sequence encoding the amino acid sequence of re-ApxIII toxin was synthesized and cloned in an expression vector, the expression vector was transfected into a host cell, and then the amino acid sequence of re-ApxIII toxin was expressed by the host cell and purified.

Alternatively, a DNA sequence encoding the amino acid sequence of re-ApxIII toxin was constructed by molecular cloning. Then, the polynucleotide sequence was inserted in an expression vector through restriction enzyme sites, and the expression vector was transfected into a host cell. Finally, the amino acid sequence of re-ApxIII toxin was expressed by the host cell and purified. In this Example, a DNA sequence encoding the epitope fragments of ApxIII toxin and a DNA sequence encoding pC3d-p31 bioadjuvant were linked with a HindIII restriction enzyme site, and the amino acid sequence of re-ApxIII toxin obtained by the cloning is SEQ ID NO: 21. The obtained re-ApxIII toxin was purified by nickel affinity chromatography and ion exchange chromatography. Then the purified re-ApxIII toxin was confirmed by sodium dodecyl sulfate polyacrylamide gel electrophoresis (SDS-PAGE) and western blot, and the results are shown in FIG. 1 (lane 3) and FIG. 2 (lane 4), respectively. The results indicate that the re-ApxIII toxin has the expected molecular weight of about 49 kDa.

### EXAMPLE 4

### Construction of Re-ApxIV Toxin

Three epitope fragments were selected from the full-length amino acid sequence of ApxIV toxin (SEQ ID NO: 38). Sequences of the three epitope fragments are listed below.

Epitope fragment ApxIV-1:

(SEQ ID NO: 66)

VIDAGAGNDTVNGGNGDDTLIGGKGNDILRGGYGADTYIFSKGHGQDIVY
EDTNNDNRAR;
Epitope fragment ApxIV-2:

(SEQ ID NO: 89)

EGKDTGFYGHAFYIERKNGGGSKNNSSGAGNSKDWGGNGHGNHRNNASDL
NKPDGNNGNNQNNGSNQDNNSDVNAPNNPGRNYD;
Epitope fragment ApxIV-3:

(SEQ ID NO: 90)

VIDAGAGNDTINGGYGDDTLIGGKGNDILKGSYGADTYIFSKGHGQDIVY
EDTNNDNRARDIDTLK.

These epitope fragments contain, but are not limited to, the following epitopes:

(SEQ ID NO: 93)

VIDAGAGNDTVNGGNGDDTLIGGKGNDILR;

(SEQ ID NO: 94)

GYGA;

(SEQ ID NO: 95)

AGAGNDTVNGGNGDDTLIGG;

(SEQ ID NO: 96)

SKGHGQDIVYEDTNNDNRAR;

(SEQ ID NO: 97)

DAGAGNDTVNGGNG;

(SEQ ID NO: 98)

DIVYEDTNNDNRAR;

(SEQ ID NO: 99)

SKGHGQDIVYEDTNNDNRAR;

(SEQ ID NO: 100)

KNGGGSKNNSSGAGNSKDWG;

(SEQ ID NO: 101)

HRNNASDLNKPDGNNGNNQN;

(SEQ ID NO: 102)

GSNQDNNSDVNAPNNPGRNY;

(SEQ ID NO: 103)

ERKNGGGSKNNSSG;

(SEQ ID NO: 104)

GNSKDWGGNGHGNH;

(SEQ ID NO: 105)

KPDGNNGNNQNNGS;

(SEQ ID NO: 106)

DNNSDVNAPNNPGR;

(SEQ ID NO: 107)

NGGGSKNNSSGAGNSKDWGG;

(SEQ ID NO: 108)

NQNNGSNQDNNSDVNAPNNP;

(SEQ ID NO: 109)

VIDAGAGNDTINGGYGDDTLIGGKGNDILK;

(SEQ ID NO: 110)

SYGA;

(SEQ ID NO: 111)

GHGQDIVYEDTNNDNRARD;

(SEQ ID NO: 112)

DAGAGNDTINGGYGDDTLIG;

(SEQ ID NO: 113)

SKGHGQDIVYEDTNNDNRAR;

(SEQ ID NO: 114)

DAGAGNDTINGGYG;

(SEQ ID NO: 115)

DIVYEDTNNDNRAR;

(SEQ ID NO: 116)

NGGYGDDTLIGGKGNDILKG;

(SEQ ID NO: 117)

SKGHGQDIVYEDTNNDNRAR.

Epitope fragments ApxIV-1, ApxIV-2, and ApxIV-3 were linked from N- to C-terminus, and each epitope fragment was linked to another epitope fragment with at least one linker having the amino acid sequence of SEQ ID NO: 26. Six (6) repeats of pC3d-p31 bioadjuvant (SEQ ID NO: 22) were added to the C-terminus of epitope fragment ApxIV-3, in which each pC3d-p31 bioadjuvant was linked to another pC3d-p31 bioadjuvant with at least one linker (SEQ ID NO: 26). The designed recombinant ApxIV toxin (re-ApxIV) has the amino acid sequence of SEQ ID NO: 91. The amino acid sequence of re-ApxIV toxin was synthesized by a peptide synthesizer. Alternatively, a polynucleotide sequence encoding the amino acid sequence of re-ApxIV toxin was synthesized and cloned in an expression vector, the expression vector was transfected into a host cell, and then the amino acid sequence of re-ApxIV toxin was expressed by the host cell and purified.

Alternatively, a DNA sequence encoding the amino acid sequence of re-ApxIV toxin was constructed by molecular cloning. Then, the polynucleotide sequence was inserted in an expression vector through restriction enzyme sites, and the expression vector was transfected into a host cell. Finally, the amino acid sequence of re-ApxIV toxin was expressed by the host cell and purified. In this Example, a DNA sequence encoding the epitope fragments of ApxIV toxin and a DNA sequence encoding pC3d-p31 bioadjuvant were linked with a HindIII restriction enzyme site, and the amino acid sequence of re-ApxIV toxin obtained by the cloning is SEQ ID NO: 92. The obtained re-ApxIV toxin was purified by nickel affinity chromatography and ion exchange chromatography. Then the purified re-ApxI toxin was confirmed by sodium dodecyl sulfate polyacrylamide gel electrophoresis (SDS-PAGE) and western blot, and the results are shown in FIG. 1 (lane 4) and FIG. 2 (lane 5), respectively. The results indicate that the re-ApxIV toxin has the expected molecular weight of about 50 kDa.

### EXAMPLE 5

### Extraction of Apx Toxin

1. Cultivation of A. pleuropneumoniae

Serotype 1 of A. pleuropneumoniae which produces ApxI, ApxII, and ApxIV (field isolated, Taiwan) and serotype 2 of A. pleuropneumoniae which produces ApxII, ApxIII, and ApxIV (field isolated, Taiwan) were separately cultivated in brain heart infusion (BHI) liquid medium containing 0.01% (v/v) β-Nicotinamide adenine dinucleotide (β-NAD) and 10% (v/v) horse serum (BD Biosciences, USA) overnight at 37° C., 5% CO₂.

### 2. Preparation of Apx Toxin

The serotypes 1 and 2 of A. pleuropneumoniae obtained above were broken by sonication (SONOPULS, Bandelin, Germany), and Apx toxin was collected by centrifugation (KUBOTA, Japan) followed by filtration of the supernatant with a 0.22 μm filter (Millipore, USA). The filtered crude extracted ApxI˜IV toxin was stored at −80° C. for further use.

3. Preparation of Inactivated A. pleuropneumoniae

Serotypes 1, 2, and 5 of A. pleuropneumoniae having the ability to produce toxin (field isolated, Taiwan) were separately cultivated in brain heart infusion (BHI) liquid medium containing 0.01% (v/v) β-Nicotinamide adenine dinucleotide (β-NAD) and 10% (v/v) horse serum (BD Biosciences, USA) overnight at 37° C., 5% CO₂. Then, the bacteria were inactivated with formaldehyde.

### EXAMPLE 6

### Preparation of Vaccine Against Porcine Pleuropneumonia

1. Preparation of Subunit Vaccine of Recombinant Apx Toxin (re-ApxI, re-ApxII, re-ApxIII, and re-ApxIV)

Each of the re-ApxI toxin obtained in Example 1 (SEQ ID NO: 8)(with a final concentration of 500 μg/ml), re-ApxII toxin obtained in Example 2 (SEQ ID NO: 16)(with a final concentration of 500 μg/ml), re-ApxIII toxin obtained in Example 3 (SEQ ID NO: 21)(with a final concentration of 500 μg/ml), and re-ApxIV toxin obtained in Example 4 (SEQ ID NO: 92)(with a final concentration of 500 μg/ml) was separately mixed with aluminum hydroxide gel (with a final concentration of 30% (v/v)) in phosphate buffered solution (PBS). The aluminum hydroxide gel was the adjuvant of each of the subunit vaccine of recombinant Apx toxin.

2. Preparation of Multivalent A. pleuropneumoniae Bacterin (App 1, 2, 5)

The inactivated serotypes 1, 2, and 5 of A. pleuropneumoniae obtained in Example 5 (with a final concentration of 1×10⁹ cfu/ml for each serotype) were mixed in phosphate buffered solution (PBS). Aluminum hydroxide gel was added to a final concentration of 30% (v/v) in the mixture as an adjuvant of the multivalent A. pleuropneumoniae bacterin (App 1, 2, 5).

3. Preparation of Multivalent Vaccine Containing A. pleuropneumoniae Bacterin and Recombinant Apx Toxin (App 1, 2, 5+re-ApxI˜III)

The re-ApxI toxin obtained in Example 1 (SEQ ID NO: 8)(with a final concentration of 20 μg/ml), re-ApxII toxin obtained in Example 2 (SEQ ID NO: 16)(with a final concentration of 20 μg/ml), re-ApxIII toxin obtained in Example 3 (SEQ ID NO: 21)(with a final concentration of 20 μg/ml), and the inactivated serotypes 1, 2, and 5 of A. pleuropneumoniae obtained in Example 5 (with a final concentration of 1×10⁹ cfu/ml for each serotype) were mixed in phosphate buffered solution (PBS). Aluminum hydroxide gel was added to a final concentration of 30% (v/v) in the mixture as an adjuvant of the multivalent vaccine containing A. pleuropneumoniae bacterin and recombinant Apx Toxin against porcine pleuropneumonia (App 1, 2, 5+re-ApxI˜III).

### EXAMPLE 7

### Efficacy Test of Subunit Vaccine of Recombinant Apx Toxin

### 1. Vaccination and Toxin Challenge in Mouse Model

Sixty (60) three-to-four-week-old healthy ICR mice (The National Laboratory Animal Center, Taiwan) were randomly divided into 6 groups (n=10 for each group), in which Group 1 was negative control, and Groups 2 to 6 were vaccination groups. All of the mice were negative for anti-A. pleuropneumoniae antibodies. Each mouse was injected intraperitoneally (i.p.) with 0.2 ml of the following substance, respectively:

Group 1: PBS solution with 30% (v/v) of aluminum hydroxide gel (PBS group);
Group 2: Subunit vaccine containing the re-ApxI toxin obtained in Example 1 (SEQ ID NO: 8) (re-ApxI group);
Group 3: Subunit vaccine containing the re-ApxII toxin obtained in Example 2 (SEQ ID NO: 16) (re-ApxII group);
Group 4: Subunit vaccine containing the re-ApxIII toxin obtained in Example 3 (SEQ ID NO: 21) (re-ApxIII group);
Group 5: Subunit vaccine containing the re-ApxIV toxin obtained in Example 4 (SEQ ID NO: 92) (re-ApxIV group); and
Group 6: The multivalent A. pleuropneumoniae bacterin obtained in Example 6 (App 1, 2, 5 group).

Fourteen (14) days after primary immunization, each mouse was boosted with the same dose of the same substance respectively. Ten (10) days after booster immunization, each mouse was challenged by injection with 0.1 ml of the crude extracted ApxI˜IV toxin obtained in Example 5, with a 90% lethal dose (LD₉₀)(extraction of 6.5×10⁹ cfu/ml of Serotype 1 and 9.65×10¹⁰ cfu/ml of Serotype 2 of A. pleuropneumoniae). Mice were observed for 10 days, and mortality of each group was recorded.

### 2. Statistical Analyses

Survival rate of each group was analyzed by Kaplan-Meier Survival Analysis: Log-Rank test. A statistically significant difference exists when the p-value is less than 0.05 (p<0.05).

### 3. Results

The results of toxin challenge are shown in Table 1. The results indicate that, comparing to negative control (Group 1, 0% survival rate), subunit vaccine containing either re-ApxI, re-ApxII, re-ApxIII, or re-ApxIV toxin (that is Group 2, 3, 4, or 5) induces a protective response against challenge with ApxI to ApxIV toxin in mice, with an increased survival rate having statistically significant difference.

TABLE 1
Survival Rate of Mice after being Challenged with ApxI to
ApxIV Toxin

| Group | Number of Mice | Survival Rate |
|---|---|---|
| Group 1 (PBS group) | 10 | 0% |
| Group 2 (re-ApxI group) | 10 | 50%* |
| Group 3 (re-ApxII group) | 10 | 50%* |
| Group 4 (re-ApxIII group) | 10 | 50%** |
| Group 5 (re-ApxIV group) | 10 | 30%* |
| Group 6 (App 1, 2, 5 group) | 10 | 20% |

*indicates a statistically significant difference exists comparing to the negative group (PBS group) (p < 0.05).
**indicates a statistically significant difference exists comparing to the negative group (PBS group) (p < 0.01).

### EXAMPLE 8

### Protection Efficacy of the Multivalent Vaccine Containing A. pleuropneumoniae Bacterin and Recombinant Apx Toxinx Against Serotype 2 of A. pleuropneumoniae (App 2)

1. Vaccination and Challenge with Serotype 2 of A. pleuropneumoniae (App 2) in Mouse Model

Forty (40) three-to-four-week-old healthy ICR mice (The National Laboratory Animal Center, Taiwan) were randomly divided into 4 groups (n=10 for each group), in which Group 1 was negative control, and Groups 2 to 4 were vaccination groups. All of the mice were negative for anti-A. pleuropneumoniae antibodies. Each mouse was injected intraperitoneally (i.p.) with 0.2 ml of the following substance, respectively:

Group 1: PBS solution with 30% (v/v) of aluminum hydroxide gel (PBS group);
Group 2: The multivalent A. pleuropneumoniae bacterin obtained in Example 6 (App 1, 2, 5 group);
Group 3: The multivalent vaccine containing A. pleuropneumoniae bacterin and recombinant Apx toxin obtained in Example 6 (App 1, 2, 5 +re-ApxI˜III group); and
Group 4: A commercial A. pleuropneumoniae bacterin containing serotypes 1, 2, 3, 4, 5, and 7 (App 1, 2, 3, 4, 5, 7 group).

Fourteen (14) days after primary immunization, each mouse was boosted with the same dose of the same substance respectively. Ten (10) days after booster immunization, each mouse was challenged by injection with 0.1 ml of a 90% lethal dose (LD₉₀)(7.5×10⁸ cfu/ml) of serotype 2 of A. pleuropneumoniae (App 2). Mice were observed for 10 days, and mortality of each group was recorded.

2. Statistical Analyses are the same as those described in Example 7.

### 3. Results

The results of challenge are shown in Table 2. The results indicate that, comparing to negative control (Group 1, 20% survival rate), each and every vaccination group (Groups 2, 3, and 4) induces a protective response against challenge with serotype 2 of A. pleuropneumoniae (App 2) in mice, with an increased survival rate having statistically significant difference. In particular, the multivalent vaccine containing A. pleuropneumoniae bacterin and the recombinant Apx toxin of the present invention (App 1, 2, 5+re-ApxI˜III group) has the best protective response with highest survival rate.

TABLE 2
Survival Rate of Mice after being Challenged with Serotype 2
of A. pleuropneumoniae (App 2)

| | Number | Survival |
|---|---|---|
| Group | of Mice | Rate |
| Group 1 (PBS group) | 10 | 20% |
| Group 2 (App 1, 2, 5 group) | 10 | 70%** |
| Group 3 (App 1, 2, 5 + re-ApxI~III group) | 10 | 80%** |
| Group 4 (Commercial App 1, 2, 3, 4, 5, 7 group) | 10 | 60%* |

*indicates a statistically significant difference exists comparing to the negative group (PBS group) (p < 0.05).
**indicates a statistically significant difference exists comparing to the negative group (PBS group) (p < 0.01).

### EXAMPLE 9

### Protection Efficacy of the Multivalent Vaccine Containing A. pleuropneumoniae Bacterin and Recombinant Apx Toxinx Against Serotype 5 of A. pleuropneumoniae (App 5)

1. Vaccination and Challenge with Serotype 5 of A. pleuropneumoniae (App 5) in Mouse Model

Fifty (50) three-to-four-week-old healthy ICR mice (The National Laboratory Animal Center, Taiwan) were randomly divided into 4 groups, in which Group 1 was negative control, and Groups 2 to 4 were vaccination groups. All of the mice were negative for anti-A. pleuropneumoniae antibodies. Each mouse was injected intraperitoneally (i.p.) with 0.2 ml of the following substance, respectively:

Group 1: PBS solution with 30% (v/v) of aluminum hydroxide gel (PBS group) (n=15);
Group 2: The multivalent A. pleuropneumoniae bacterin obtained in Example 6 (App 1, 2, 5 group) (n=15);
Group 3: The multivalent vaccine containing A. pleuropneumoniae bacterin and recombinant Apx toxin obtained in Example 6 (App 1, 2, 5+re-ApxI˜III group) (n=10); and
Group 4: A commercial A. pleuropneumoniae bacterin containing serotypes 1, 2, 3, 4, 5, and 7 (App 1, 2, 3, 4, 5, 7 group) (n=10).

Fourteen (14) days after primary immunization, each mouse was boosted with the same dose of the same substance respectively. Serum samples were collected 24 hours prior to primary immunization and 10 days after booster immunization. All the serum samples were tested by ELISA. Ten (10) days after booster immunization and blood sampling, each mouse was challenged by injection with 0.1 ml of a 90% lethal dose (LD₉₀)(7.32×10⁸ cfu/ml) of serotype 5 of A. pleuropneumonias (App 5). Mice were observed for 10 days, and mortality of each group was recorded.

### 2. Enzyme-Linked Immunosorbent Assay (ELISA) of Toxin Antibodies

Recombinant ApxII toxin (re-ApxII' SEQ ID NO: 16) was coated on 96-well ELISA plates as antigen for 16 hours at 4° C. Uncoated antigen was removed, and the ELISA plates were washed 3 times with wash buffer (0.9% NaCl and 0.1% Tween 20) and air-dried. To block the ELISA plates, blocking solution (wash buffer containing 1% BSA) was added to the ELISA plates, and then the ELISA plates were incubated for an hour at room temperature. After that, the ELISA plates were washed with wash buffer. Mouse serum samples were diluted with PBS and added to the wells of the ELISA plates, and the plates were incubated for an hour at room temperature. After incubation, the serum samples were removed, and the plates were washed with PBS. Secondary antibody conjugated to horseradish peroxidase (HRP)(goat anti-mouse antibody conjugated HRP, Gene Tex Inc., USA) was diluted 5000-fold with blocking solution and then added to the wells (100 μl/well). After incubating for an hour at room temperature, the secondary antibody was removed, and the plates were washed with wash buffer. For visualization of results, 100 μl of 3,3′,5,5′-tetramethylbenzidine (TMB, KPL Inc., USA) was added to each well, and the plates were incubated for 10 minutes in dark. Optical density at 650 nm (OD_{650 nm}) was read with an ELISA plate reader (SpectraMax® M2/M2 ELISA Reader, Molecular Devices, LLC., USA).

### 3. Statistical Analyses

Survival rate of each group was analyzed by Kaplan-Meier Survival Analysis: Log-Rank test. A statistically significant difference exists when the p-value is less than 0.05 (p<0.05). Results of ELISA were analyzed by Student Newman-Keuls Method. A statistically significant difference exists when the p-value is less than 0.05 (p<0.05).

### 4. Results

The results of challenge are shown in Table 3. The results indicate that, comparing to negative control (Group 1, 6.7% survival rate), each and every vaccination group (Groups 2, 3, and 4) induces a protective response against challenge with serotype 5 of A. pleuropneumoniae (App 5) in mice, with an increased survival rate having statistically significant difference. In particular, the multivalent vaccine containing A. pleuropneumoniae bacterin and the recombinant Apx toxin of the present invention (App 1, 2, 5+re-ApxI˜III group) has the best protective response with highest survival rate.

TABLE 3
Survival Rate of Mice after being Challenged with Serotype 5 of
A. pleuropneumoniae (App 5)

| | Number | Survival |
|---|---|---|
| Group | of Mice | Rate |
| Group 1 (PBS group) | 15 | 6.7% |
| Group 2 (App 1, 2, 5 group) | 15 | 26.7%* |
| Group 3 (App 1, 2, 5 + re-ApxI~III group) | 10 | 70%** |
| Group 4 (Commercial App 1, 2, 3, 4, 5, 7 group) | 10 | 60%* |

*indicates a statistically significant difference exists comparing to the negative group (PBS group) (p < 0.05).
**indicates a statistically significant difference exists comparing to the negative group (PBS group) (p < 0.01).

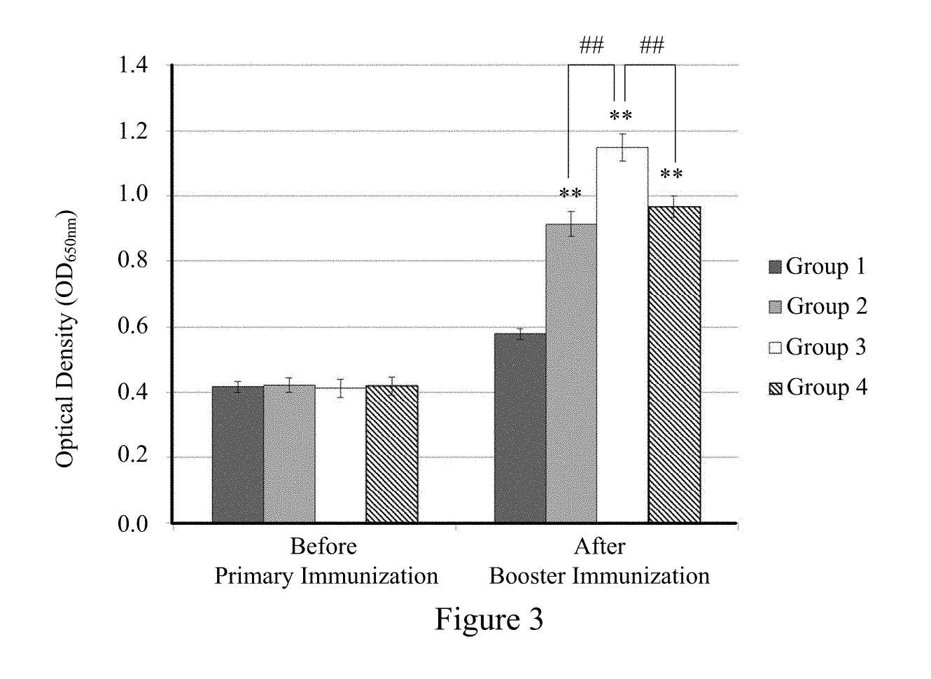

ELISA results are shown in FIG. 3. Ten (10) days after booster immunization, the multivalent vaccine containing A. pleuropneumoniae bacterin and the recombinant Apx toxin of the present invention (Group 3, i.e. App 1, 2, 5+re-ApxI˜III group) induced significantly higher level of serum antibodies against Apx toxin in mice than negative control (Group 1, i.e. PBS group, p<0.01), the multivalent A. pleuropneumoniae bacterin (Group 2, i.e. App 1, 2, 5 group, p<0.01), and the commercial A. pleuropneumoniae bacterin containing serotypes 1, 2, 3, 4, 5, and 7 (Group 4, i.e. App 1, 2, 3, 4, 5, 7 group, p<0.01) did. Therefore, the results indicated that the multivalent vaccine containing A. pleuropneumoniae bacterin and the recombinant Apx toxin of the present invention is able to effectively induce immune responses against Apx toxin in mice and has immunogenicity significantly better than the commercial A. pleuropneumoniae vaccine.

### EXAMPLE 10

### Preparation of Anti-ApxI to ApxIV Polyclonal Antibodies

Each of the re-ApxI toxin obtained in Example 1 (SEQ ID NO: 8), re-ApxII toxin obtained in Example 2 (SEQ ID NO: 16), re-ApxIII toxin obtained in Example 3 (SEQ ID NO: 21), and re-ApxIV toxin obtained in Example 4 (SEQ ID NO: 92) was separately mixed with Freund's complete adjuvant (FCA, Sigma, USA). Each of the mixture was primarily inoculated intraperitoneally into Balb/c mice (0.2 ml/mouse), and a second immunization was performed after 3 weeks. After one week, serum of the inoculated mice was collected as anti-Apx1/2/3/4 polyclonal antibodies. The polyclonal antibodies are used for western blot.

The foregoing description of the exemplary embodiments of the invention has been presented only for the purposes of illustration and description and is not intended to be exhaustive or to limit the invention to the precise forms disclosed. Many modifications and variations are possible in light of the above teaching.

What is claimed is:

1. A recombinant Actinobacillus pleuropneumonias toxin (re-Apx) represented by formula (I):
(A)ₘ-(C3d fragment)ₙ (I)
wherein
each A is an individual epitope of an Actinobacillus pleuropneumonias toxin (Apx);
each C3d fragment is an individual unit of the amino acid sequence of complement C3d and is independently selected from the group consisting of SEQ ID NOs: 22, 23, 24, and 25;
m is an integer from 1 to about 30; and
n is an integer from 0 to about 10.

2. The recombinant Actinobacillus pleuropneumonias toxin (re-Apx) of claim 1, wherein each A is linked to another A with a linker, and each linker is independently selected from the group consisting of Gly-Gly, Gly-Ser, and SEQ ID NOs: 26, 27, 28, 29, 30, 31, 32, 33, 34, 35, and 36.

3. The recombinant Actinobacillus pleuropneumonias toxin (re-Apx) of claim 1, wherein each C3d fragment is linked to another C3d fragment with a linker, and each linker is independently selected from the group consisting of Gly-Gly, Gly-Ser, and SEQ ID NOs: 26, 27, 28, 29, 30, 31, 32, 33, 34, 35, and 36.

4. The recombinant Actinobacillus pleuropneumonias toxin (re-Apx) of claim 1, wherein the A next to a C3d fragment is linked to the C3d fragment with a linker, and the linker is selected from the group consisting of Gly-Gly, Gly-Ser, and SEQ ID NOs: 26, 27, 28, 29, 30, 31, 32, 33, 34, 35, and 36.

5. The recombinant Actinobacillus pleuropneumonias toxin (re-Apx) of claim 1, wherein the Actinobacillus pleuropneumonias toxin (Apx) is ApxI toxin, the recombinant Actinobacillus pleuropneumonias toxin is re-ApxI toxin, and each A is independently selected from the group consisting of the amino acid sequences of SEQ ID NOs: 2, 3, 4, 5, 6, 37, 39, 40, 41, 42, 43, 44, 45, 46, 47, 48, 49, 50, and 51.

6. The recombinant Actinobacillus pleuropneumonias toxin (re-Apx) of claim 5, wherein the re-ApxI toxin comprises the amino acid sequences of SEQ ID NOs: 7 or 8.

7. The recombinant Actinobacillus pleuropneumonias toxin (re-Apx) of claim 1, wherein the Actinobacillus pleuropneumonias toxin (Apx) is ApxII toxin, the recombinant Actinobacillus pleuropneumonias toxin is re-ApxII toxin, and each A is independently selected from the group consisting of the amino acid sequences of SEQ ID NOs: 10, 11, 12, 13, 14, 52, 53, 54, 55, 56, 57, 58, 59, 60, 61, 62, 63, 64, 65, 67, and 68.

8. The recombinant Actinobacillus pleuropneumonias toxin (re-Apx) of claim 7, wherein the re-ApxII toxin comprises the amino acid sequences of SEQ ID NOs: 15 or 16.

9. The recombinant Actinobacillus pleuropneumonias toxin (re-Apx) of claim 1, wherein the Actinobacillus pleuropneumonias toxin (Apx) is ApxIII toxin, the recombinant Actinobacillus pleuropneumonias toxin is re-ApxIII toxin, and each A is independently selected from the group consisting of the amino acid sequences of SEQ ID NOs: 18, 19, 69, 70, 71, 72, 73, 74, 75, 76, 77, 78, 79, 80, 81, 82, 83, 84, 85, 86, 87, and 88.

10. The recombinant Actinobacillus pleuropneumonias toxin (re-Apx) of claim 9, wherein the re-ApxIII toxin comprises the amino acid sequences of SEQ ID NOs: 20 or 21.

11. The recombinant Actinobacillus pleuropneumonias toxin (re-Apx) of claim 1, wherein the Actinobacillus pleuropneumonias toxin (Apx) is ApxIV toxin, the recombinant Actinobacillus pleuropneumonias toxin is re-ApxIV toxin, and each A is independently selected from the group consisting of the amino acid sequences of SEQ ID NOs: 66, 89, 90, 93, 94, 95, 96, 97, 98, 99, 100, 101, 102, 103, 104, 105, 106, 107, 108, 109, 110, 111, 112, 113, 114, 115, 116, and 117.

12. The recombinant Actinobacillus pleuropneumonias toxin (re-Apx) of claim 11, wherein the re-ApxIV toxin comprises the amino acid sequences of SEQ ID NOs: 91 or 92.

13. A polynucleotide comprising a nucleotide sequence encoding the recombinant Actinobacillus pleuropneumonias toxin (re-Apx) of claim 1.

14. An immunogenic composition against porcine pleuropneumonia, comprising the recombinant Actinobacillus pleuropneumonias toxin (re-Apx) of claim 1 and a pharmaceutically acceptable vehicle.

15. The immunogenic composition against porcine pleuropneumonia of claim 14, wherein the recombinant Actinobacillus pleuropneumonias toxin (re-Apx) is selected from at least one of the group consisting of re-ApxI toxin and each A is independently selected from the group consisting of the amino acid sequences of SEQ ID NOs: 2, 3, 4, 5, 6, 37, 39, 40, 41, 42, 43, 44, 45, 46, 47, 48, 49, 50, and 51, re-ApxII toxin and each A is independently selected from the group consisting of the amino acid sequences of SEQ ID NOs: 10, 11, 12, 13, 14, 52, 53, 54, 55, 56, 57, 58, 59, 60, 61, 62, 63, 64, 65, 67, and 68, re-ApxIII toxin and each A is independently selected from the group consisting of the amino acid sequences of SEQ ID NOs: 18, 19, 69, 70, 71, 72, 73, 74, 75, 76, 77, 78, 79, 80, 81, 82, 83, 84, 85, 86, 87, and 88, and re-ApxIV toxin and each A is independently selected from the group consisting of the amino acid sequences of SEQ ID NOs: 66, 89, 90, 93, 94, 95, 96, 97, 98, 99, 100, 101, 102, 103, 104, 105, 106, 107, 108, 109, 110, 111, 112, 113, 114, 115, 116, and 117.

16. The immunogenic composition against porcine pleuropneumonia of claim 14, further comprising at least one serotype of A. pleuropneumonias.

17. The immunogenic composition against porcine pleuropneumonia of claim 14, further comprising at least one pathogen antigen selected from the group consisting of antigen of porcine circovirus type 2 (PCV2), antigen of Swine influenza virus (SIV), antigen of porcine reproductive and respiratory syndrome virus (PRRSV), antigen of mycoplasma, antigen of porcine parvovirus (PPV), antigen of erysipelas, antigen of Bordetella bronchiseptica, antigen of Pasteurella multocida, and antigen of pseudorabies (Aujeszky's disease) virus.

18. A method of protecting an animal against porcine pleuropneumonia, comprising administering the immunogenic composition of claim 14 to the animal to increase immunity against porcine pleuropneumonia in the animal.

19. An anti-Actinobacillus pleuropneumonias toxin (Apx) antibody derived from the recombinant Actinobacillus pleuropneumonias toxin (re-Apx) of claim 1.

20. The anti-Actinobacillus pleuropneumonias toxin (Apx) antibody of claim 19, wherein the antibody comprises at least one of a monoclonal antibody, a polyclonal antibody, and a genetically engineered antibody.

21. A test kit for porcine pleuropneumonia, comprising a detecting unit which is at least one of the recombinant Actinobacillus pleuropneumonias toxin (re-Apx) of claim 1 and an anti-Actinobacillus pleuropneumonias toxin (Apx) antibody derived from the recombinant Actinobacillus pleuropneumonias toxin (re-Apx) of claim 1.

22. The test kit of claim 21, wherein the antibody comprises at least one of a monoclonal antibody, a polyclonal antibody, and a genetically engineered antibody.


SEQUENCE LISTING (117 sequences)

SEQ ID NO: 1 - protein, 1022 aa, Actinobacillus pleuropneumoniae
MANSQLDRVKGLIDSLNQHTKSAAKSGAGALKNGLGQVKQAGQKLILYIPKDYQASTGSSLNDLVKAAEALGIEVHRSEKNGTALAKELFGTTEKLLGFSERGIALFAPQFDKLLNKNQKLSKSLGGSSEALGQRLNKTQTALSALQSFLGTAIAGMDLDSLLRRRRNGEDVSGSELAKAGVDLAAQLVDNIASATGTVDAFAEQLGKLGNALSNTRLSGLASKLNNLPDLSLAGPGFDAVSGILSVVSASFILSNKDADAGTKAAAGIEISTKILGNIGKAVSQYIIAQRVAAGLSTTAATRWFNRSVVALAISPLSFLNVADKFERAKQLEQYSERFKKFGYEGDSLLASFYRETGAIEAALTTINSVLSAAPAGVGAAATGSLVGAPVAALVSAITGIISGILDASKQAIFERVATKLANKIDEWEKKHGKNYFENGYDARHSAFLEDTFELLSQYNKEYSVERVVAITQQRWDVNIGELAGITRKGADAKSGKAYVDFFEEGKLLEKDPDRFDKKVFDPLEGKIDLSSINKTTLLKFITPAFTAGEEIRERKQTGKYEYMTELFVKGKEKWVVTGVQSHNAIYDYTNLIQLAIDKKGEKRQVTIESHLGEKNDRIYLSSGSSIEYAGNGHDVAYYDKTDTGYLTFDGQSAQKAGEYIVTKELKADVKVLKEVVKTQDISVGKRSEKLEYRDYELSPFELGNGIRAKDELHSVEEIIGSNRKDKFFGSRFTDIFHGAKGDDEIYGNDGHDILYGDDGNDVIHGGDGNDHLVGGNGNDRLIGGKGNNFLNGGDGDDELQVFECQYNVLLGGAGNDILYGSDGTNLFDGGVGNDKIYGGLGKDIYRYSKEYGRHIIIEKGGDDDTLLLSDLSFKDVGFIRIGDDLLVNKRIGGTLYYHEDYNGNALTIKDWFKEGKEGQNNKVEKIVDKDGAYVLSQYLTELTAPGRGINYFNGLEEKLYYGEGYNALPQLRKDIEQIISSTGALTGEHGQVLVGAGGPLAYSNSPNSIPNAFSNYLTQSA

SEQ ID NO: 2 - protein, 16 aa, Actinobacillus pleuropneumoniae
ELAGITRKGADAKSGK

SEQ ID NO: 3 - protein, 8 aa, Actinobacillus pleuropneumoniae
PAGVGAAA

SEQ ID NO: 4 - protein, 24 aa, Actinobacillus pleuropneumoniae
DILYGSDGTNLFDGGVGNDKIYGG

SEQ ID NO: 5 - protein, 26 aa, Actinobacillus pleuropneumoniae
EHGQVLVGAGGPLAYSNSPNSIPNAF

SEQ ID NO: 6 - protein, 97 aa, Actinobacillus pleuropneumoniae
RAKDELHSVEEIIGSNRKDKFFGSRFTDIFHGAKGDDEIYGNDGHDILYGDDGNDVIHGGDGNDHLVGGNGNDRLIGGKGNNFLNGGDGDDELQVFE

SEQ ID NO: 7 - protein, 410 aa, Artificial Sequence
  Recombinant ApxI toxin
ELAGITRKGADAKSGKGSTAPAGVGAAAGSTAGSTADILYGSDGTNLFDGGVGNDKIYGGGSTAEHGQVLVGAGGPLAYSNSPNSIPNAFGSTARAKDELHSVEEIIGSNRKDKFFGSRFTDIFHGAKGDDEIYGNDGHDILYGDDGNDVIHGGDGNDHLVGGNGNDRLIGGKGNNFLNGGDGDDELLQVFEGSTAGSTAFLNKLLSTAKERNRWEEPGQKLYNVEATSYAGSTAFLNKLLSTAKERNRWEEPGQKLYNVEATSYAGSTAFLNKLLSTAKERNRWEEPGQKLYNVEATSYAGSTAFLNKLLSTAKERNRWEEPGQKLYNVEATSYAGSTAFLNKLLSTAKERNRWEEPGQKLYNVEATSYAGSTAFLNKLLSTAKERNRWEEPGQKLYNVEATSYAGSTA

SEQ ID NO: 8 - protein, 412 aa, Artificial Sequence
  Recombinant ApxI toxin with a HindIII site
ELAGITRKGADAKSGKGSTAPAGVGAAAGSTAGSTADILYGSDGTNLFDGGVGNDKIYGGGSTAEHGQVLVGAGGPLAYSNSPNSIPNAFGSTARAKDELHSVEEIIGSNRKDKFFGSRFTDIFHGAKGDDEIYGNDGHDILYGDDGNDVIHGGDGNDHLVGGNGNDRLIGGKGNNFLNGGDGDDELLQVFEGSTAKLGSTAFLNKLLSTAKERNRWEEPGQKLYNVEATSYAGSTAFLNKLLSTAKERNRWEEPGQKLYNVEATSYAGSTAFLNKLLSTAKERNRWEEPGQKLYNVEATSYAGSTAFLNKLLSTAKERNRWEEPGQKLYNVEATSYAGSTAFLNKLLSTAKERNRWEEPGQKLYNVEATSYAGSTAFLNKLLSTAKERNRWEEPGQKLYNVEATSYAGSTA

SEQ ID NO: 9 - protein, 956 aa, Actinobacillus pleuropneumoniae
MSKITLSSLKSSLQQGLKNGKNKLNQAGTTLKNGLTQTGHSLQNGAKKLILYIPQGYDSGQGNGVQDLVKAANDLGIEVWREERSNLDIAKTSFDTTQKILGFTDRGIVLFAPQLDNLLKKNPKIGNTLGSASSISQNIGKANTVLGGIQSILGSVLSGVNLNELLQNKDPNQLELAKAGLELTNELVGNIASSVQTVDAFAEQISKLGSHLQNVKGLGGLSNKLQNLPDLGKASLGLDIISGLLSGASAGLILADKEASTEKKAAAGVEFANQIIGNVTKAVSSYILAQRVASGLSSTGPVAALIASTVALAVSPLSFLNVADKFKQADLIKSYSERFQKLGYDGDRLLADFHRETGTIDASVTTINTALAAISGGVGAASAGSLVGAPVALLVAGVTGLITTILEYSKQAMFEHVANKVHDRIVEWEKKHNKNYFEQGYDSRHLADLQDNMKFLINLNKELQAERVVAITQQRWDNQIGDLAAISRRTDKISSGKAYVDAFEEGQHQSYDSSVQLDNKNGIINISNTNRKTQSVLFRTPLLTPGEENRERIQEGKNSYITKLHIQRVDSWTVTDGDASSSVDFTNVVQRIAVKFDDAGNIIESKDTKIIANLGAGNDNVFVGSSTTVIDGGDGHDRVHYSRGEYGALVIDATAETEKGSYSVKRYVGDSKALHETIATHPTNVGNREEKIEYRREDDRFHTGYTVTDSLKSVEEIIGSQFNDIFKGSQFDDVFHGGNGVDTIDGNDGDDHLFGGAGDDVIDGGNGNNFLVGGTGNDIISGGKDNDIYVHKTGDGNDSITDSGGQDKLAFSDVNLKDLTFKKVDSSLEIINQKGEKVRIGNWFLKNDLASTVANYKATNDRKIEEIIGKGGERITSKQVDKLIKEGNNQISAKALSKVGNDYNTSKDRQNVSNSLAKLISSVESFTSSSNFRNNLGAYVPSSINVSNNIQLARAA

SEQ ID NO: 10 - protein, 72 aa, Actinobacillus pleuropneumoniae
GNGVDTIDGNDGDDHLFGGAGDDVIDGGNGNNFLVGGTGNDIISGGKDNDIYVHKTGDGNDSITDSGGQDKL

SEQ ID NO: 11 - protein, 15 aa, Actinobacillus pleuropneumoniae
KVGNDYNTSKDRQNV

SEQ ID NO: 12 - protein, 41 aa, Actinobacillus pleuropneumoniae
LTPGEENRERIQEGKNSYITKLHIQRVDSWTVTDGDASSSV

SEQ ID NO: 13 - protein, 20 aa, Actinobacillus pleuropneumoniae
ILYIPQGYDSGQGNGVQDLV

SEQ ID NO: 14 - protein, 24 aa, Actinobacillus pleuropneumoniae
ATHPTNVGNREEKIEYRREDDRFH

SEQ ID NO: 15 - protein, 410 aa, Artificial Sequence
  Recombinant ApxII toxin
GNGVDTIDGNDGDDHLFGGAGDDVIDGGNGNNFLVGGTGNDIISGGKDNDIYVHKTGDGNDSITDSGGQDKLGSTAKVGNDYNTSKDRQNVGSTAGSTALTPGEENRERIQEGKNSYITKLHIQRVDSWTVTDGDASSSVGSTAILYIPQGYDSGQGNGVQDLVGSTAATHPTNVGNREEKIEYRREDDRFHGSTAGSTAFLNKLLSTAKERNRWEEPGQKLYNVEATSYAGSTAFLNKLLSTAKERNRWEEPGQKLYNVEATSYAGSTAFLNKLLSTAKERNRWEEPGQKLYNVEATSYAGSTAFLNKLLSTAKERNRWEEPGQKLYNVEATSYAGSTAFLNKLLSTAKERNRWEEPGQKLYNVEATSYAGSTAFLNKLLSTAKERNRWEEPGQKLYNVEATSYAGSTA

SEQ ID NO: 16 - protein, 412 aa, Artificial Sequence
  Recombinant ApxII toxin with a HindIII site
GNGVDTIDGNDGDDHLFGGAGDDVIDGGNGNNFLVGGTGNDIISGGKDNDIYVHKTGDGNDSITDSGGQDKLGSTAKVGNDYNTSKDRQNVGSTAGSTALTPGEENRERIQEGKNSYITKLHIQRVDSWTVTDGDASSSVGSTAILYIPQGYDSGQGNGVQDLVGSTAATHPTNVGNREEKIEYRREDDRFHGSTAKLGSTAFLNKLLSTAKERNRWEEPGQKLYNVEATSYAGSTAFLNKLLSTAKERNRWEEPGQKLYNVEATSYAGSTAFLNKLLSTAKERNRWEEPGQKLYNVEATSYAGSTAFLNKLLSTAKERNRWEEPGQKLYNVEATSYAGSTAFLNKLLSTAKERNRWEEPGQKLYNVEATSYAGSTAFLNKLLSTAKERNRWEEPGQKLYNVEATSYAGSTA

SEQ ID NO: 17 - protein, 1052 aa, Actinobacillus pleuropneumoniae
MSTWSSMLADLKKRAEEAKRQAKKGYDVTKNGLQYGVSQAKLQALAAGKAVQKYGNKLVLVIPKEYDGSVGNGFFDLVKAAEELGIQVKYVNRNELEVAHKSLGTADQFLGLTERGLTLSAPQLDQFLQKHSKISNVVGSSTGDAVSKLAKSQTIISGIQSVLGTVLAGINLNEAIISGGSELELAEAGVSLASELVSNIAKGTTTIDAFTTQIQNFGKLVENAKGLGGVGRQLQNISGSALSKTGLGLDIISSLLSGVTASFALANKNASTSTKVAAGFELSNQVIGGITKAVSSYILAQRLAAGLSTTGPAAALIASSISLAISPLAFLRVADNFNRSKEIGEFAERFKKLGYDGDKLLSEFYHEAGTIDASITTISTALSAIAAGTAAASAGALVGAPITLLVTGITGLISGILEFSKQPMLDHVASKIGNKIDEWEKKYGKNYFENGYDARHKAFLEDSFSLLSSFNKQYETERAVLITQQRWDEYIGELAGITGKGDKLSSGKAYVDYFQEGKLLEKKPDDFSKVVFDPTKGEIDISNSQTSTLLKFVTPLLTPGTESRERTQTGKYEYITKLVVKGKDKWVVNGVKDKGAVYDYTNLIQHAHISSSVARGEEYREVRLVSHLGNGNDKVFLAAGSAEIHAGEGHDVVYYDKTDTGLLVIDGTKATEQGRYSVTRELSGATKILREVIKNQKSAVGKREETLEYRDYELTQSGNSNLKAHDELHSVEEIIGSNQRDEFKGSKFRDIFHGADGDDLLNGNDGDDILYGDKGNDELRGDNGNDQLYGGEGNDKLLGGNGNNYLSGGDGNDELQVLGNGFNVLRAGKGDDKLYGSSGSDLLDGGEGNDYLEGGDGSDFYVYRSTSGNHTIYDQGKSSDLDKLYLSDFSFDRLLVEKVDDNLVLRSNESSHNNRVLTIKDWFKEGNKYNHKIEQIVDKNGRKLTAENLGTYFKNAPKADNLLNYATKEDQNESNLSSLKTELSKIITNAGNFGVAKQGNTGINTAALNNEVNKIISSANTFATSQLGGSGMGTLPSTNVNSMMLGNLARAA

SEQ ID NO: 18 - protein, 127 aa, Actinobacillus pleuropneumoniae
ADGDDLLNGNDGDDILYGDKGNDELRGDNGNDQLYGGEGNDKLLGGNGNNYLSGGDGNDELQVLGNGFNVLRAGKGDDKLYGSSGSDLLDGGEGNDYLEGGDGSDFYVYRSTSGNHTIYDQGKSSDL

SEQ ID NO: 19 - protein, 80 aa, Actinobacillus pleuropneumoniae
GELAGITGKGDKLSSGKAYVDYFQEGKLLEKKPDDFSKVVFDPTKGEIDISNSQTSTLLKFVTPLLTPGTESRERTQTGK

SEQ ID NO: 20 - protein, 433 aa, Artificial Sequence
  Recombinant ApxIII toxin
ADGDDLLNGNDGDDILYGDKGNDELRGDNGNDQLYGGEGNDKLLGGNGNNYLSGGDGNDELQVLGNGFNVLRAGKGDDKLYGSSGSDLLDGGEGNDYLEGGDGSDFYVYRSTSGNHTIYDQGKSSDLGSTAGSTAGELAGITGKGDKLSSGKAYVDYFQEGKLLEKKPDDFSKVVFDPTKGEIDISNSQTSTLLKFVTPLLTPGTESRERTQTGKGSTAGSTAFLNKLLSTAKERNRWEEPGQKLYNVEATSYAGSTAFLNKLLSTAKERNRWEEPGQKLYNVEATSYAGSTAFLNKLLSTAKERNRWEEPGQKLYNVEATSYAGSTAFLNKLLSTAKERNRWEEPGQKLYNVEATSYAGSTAFLNKLLSTAKERNRWEEPGQKLYNVEATSYAGSTAFLNKLLSTAKERNRWEEPGQKLYNVEATSYAGSTA

SEQ ID NO: 21 - protein, 435 aa, Artificial Sequence
  Recombinant ApxIII with a HindIII site
ADGDDLLNGNDGDDILYGDKGNDELRGDNGNDQLYGGEGNDKLLGGNGNNYLSGGDGNDELQVLGNGFNVLRAGKGDDKLYGSSGSDLLDGGEGNDYLEGGDGSDFYVYRSTSGNHTIYDQGKSSDLGSTAGSTAGELAGITGKGDKLSSGKAYVDYFQEGKLLEKKPDDFSKVVFDPTKGEIDISNSQTSTLLKFVTPLLTPGTESRERTQTGKGSTAKLGSTAFLNKLLSTAKERNRWEEPGQKLYNVEATSYAGSTAFLNKLLSTAKERNRWEEPGQKLYNVEATSYAGSTAFLNKLLSTAKERNRWEEPGQKLYNVEATSYAGSTAFLNKLLSTAKERNRWEEPGQKLYNVEATSYAGSTAFLNKLLSTAKERNRWEEPGQKLYNVEATSYAGSTAFLNKLLSTAKERNRWEEPGQKLYNVEATSYAGSTA

SEQ ID NO: 22 - protein, 31 aa, Sus scrofa
  ref <308>: DATABASE ACCESSION NUMBER: GenBank: ADG26759
  ref <309>: DATABASE ENTRY DATE: 2010-05-08
  ref <313>: RELEVANT RESIDUES IN SEQ ID NO: (201)..(231)
FLNKLLSTAKERNRWEEPGQKLYNVEATSYA

SEQ ID NO: 23 - protein, 296 aa, Sus scrofa
  ref <308>: DATABASE ACCESSION NUMBER: GenBank: ADG26759
  ref <309>: DATABASE ENTRY DATE: 2010-05-08
  ref <313>: RELEVANT RESIDUES IN SEQ ID NO: (1)..(296)
TPSGCGKQNMIGMTPTVIAVHYLDSTEQWEKFGLEKRQEALELIKKGYTQQLAFRQKNSAFAAFQDRLSSTWLTAYVVKVFAMAANLIAIDSQVLCGAVKWLILEKQKPDGVFEENGPVIHQEMIGGFKNTEEKDVSLTAFVLIALQEAKDICEPQVNSLLRSINKARDFLADYYLELKRPYTVAIAGYALALSDKLDEPFLNKLLSTAKERNRWEEPGQKLYNVEATSYALLALLVVKDFDSVPPIVRWLNEQRYYGGGYGSTQATFMVFQALAQYQKDVPDHKDLNLDVSIHLP

SEQ ID NO: 24 - protein, 28 aa, Mus musculus
  ref <308>: DATABASE ACCESSION NUMBER: GenBank: ADL70201
  ref <309>: DATABASE ENTRY DATE: 2010-08-18
  ref <313>: RELEVANT RESIDUES IN SEQ ID NO: (211)..(238)
KFLNTAKDRNRWEEPDQQLYNVEATSYA

SEQ ID NO: 25 - protein, 312 aa, Mus musculus
  ref <308>: DATABASE ACCESSION NUMBER: GenBank: ADL70201
  ref <309>: DATABASE ENTRY DATE: 2010-08-18
  ref <313>: RELEVANT RESIDUES IN SEQ ID NO: (1)..(312)
RLKHLIVTPAGCGEQNMIGMTPTVIAVHYLDQTEQWEKFGIEKRQEALELIKKGYTQQLAFKQPSSAYAAFNNRPPSTWLTAYVVKVFSLAANLIAIDSHVLCGAVKWLILEKQKPDGVFQEDGPVIHQEMIGGFRNAKEADVSLTAFVLIALQEARDICEGQVNSLPGSINKAGEYIEASYMNLQRPYTVAIAGYALALMNKLEEPYLGKFLNTAKDRNRWEEPDQQLYNVEATSYALLALLLLKDFDSVPPVVRWLNEQRYYGGGYGSTQATFMVFQALAQYQTDVPDHKDLNMDVSFHLPSCSSATTFR

SEQ ID NO: 26 - protein, 4 aa, Artificial Sequence
  Amino acid sequence of GSTA linker
GSTA

SEQ ID NO: 27 - protein, 9 aa, Artificial Sequence
  Amino acid sequence of GS linker
GGGGSGGGG

SEQ ID NO: 28 - protein, 14 aa, Artificial Sequence
  Amino acid sequence of GSGGG linker
GSGGGGSGGGGSGS

SEQ ID NO: 29 - protein, 6 aa, Artificial Sequence
  Amino acid sequence of (GS)3 linker
GSGSGS

SEQ ID NO: 30 - protein, 15 aa, Artificial Sequence
  amino acid sequence of (GGGGS)3 linker
GGGGSGGGGSGGGGS

SEQ ID NO: 31 - protein, 4 aa, Artificial Sequence
  amino acid sequence of GGSG linker
GGSG

SEQ ID NO: 32 - protein, 8 aa, Artificial Sequence
  Amino acid sequence of (GGSG)2 linker
GGSGGGSG

SEQ ID NO: 33 - protein, 12 aa, Artificial Sequence
  amino acid sequence of (GGSG)3 linker
GGSGGGSGGGSG

SEQ ID NO: 34 - protein, 10 aa, Artificial Sequence
  amino acid sequence of GENLYFQSGG linker
GENLYFQSGG

SEQ ID NO: 35 - protein, 36 aa, Artificial Sequence
  amino acid sequence of GAST linker
GGSAGGSGSGSSGGSSGASGTGTAGGTGSGSGTGSG

SEQ ID NO: 36 - protein, 36 aa, Artificial Sequence
  amino acid sequence of SEG linker
GGSGGGSEGGGSEGGGSEGGGSEGGGSEGGGSGGGS

SEQ ID NO: 37 - protein, 10 aa, Actinobacillus pleuropneumoniae
RKGADAKSGK

SEQ ID NO: 38 - protein, 1951 aa, Actinobacillus pleuropneumoniae
MTKLTMQDVTNLYLYKTKTLPKDRLDDSLISEIGKGDDDIDRKEFMVGPGRFVTADNFSVVRDFFNAGKSRIIAPQVPPIRSQQEKILVGLKPGKYSKAQILEMLGYTKGGEVVNGMFAGEVQTLGFYDDGKGDLLERAYIWNTTGFKMSDNAFFVIEESGKRYIENFGIEPLGKQEDFDFVGGFWSNLVNRGLESIIDPSGIGGTVNLNFTGEVETYTLDETRFKAEAAKKSHWSLVNAAKVYGGLDQIIKKLWDSGSIKHLYQDKDTGKLKPIIYGTAGNDSKIEGTKITRRIAGKEVTLDIANQKIEKGVLEKLGLSVSGSDIIKLLFGALTPTLNRMLLSQLIQSFSDSLAKLDNPLAPYTKNGVVYVTGKGNDVLKGTEHEDLFLGGEGNDTYYARVGDTIEDADGKGKVYFVREKGVPKADPKRVEFSEYITKEEIKEVEKGLLTYAVLENYNWEEKTATFAHATMLNELFTDYTNYRYEVKGLKLPAVKKLKSPLVEFTADLLTVTPIDENGKALSEKSITVKNFKNGDLGIRLLDPNSYYYFLEGKDTGFYGHAFYIERKNGGGSKNNSSGAGNSKDWGGNGHGNHRNNASDLNKPDGNNGNNQNNGSNQDNNSDVNAPNNPGRNYDIYDPLALDLDGDGLETVSMNGRQGALFDHEGKGIRTATGWLAADDGFLVLDRNQDGIINDISELFSNKNQLSDGSISAHGFAALADLDTNQDQRIDQNDKLFSKLQIWRDLNQNGFSEANELFSLESLNIKSLHTAYEERNDFLAGNNILAQLGKYEKTDGTFAQMGDLNFSFNPFYSRFTEALNLTEQQRRTINLTGTGRVRDLREAAALSEELAALLQQYTKASDFQAQRELLPAILDKWAATDLQYQHYDKTLLKTLESTDSSASVVRVTPSQLSSIRNVKHDPTVMQNFEQSKAKIATLNSLYGLNIDQLYYTTDKDIRYITDKVNNMYQTTVELAYRSLLLQTRLKKYVYSVNAKQFEGKWVADYSRTEALFNSTFKQSPENALYDLSEYLSFFNDPTEWKEGLLLLSRYIDYAKAQGFYENWATTSNLTIARLREAGVIFAESTDLKGDEKNNILLGSQKDNNLSGSAGDDLLIGGEGNDTLKGSYGADTYLFSKGHGQDVIYEYSDSANSKSDIDTLKFTDINYAEVKFRRVGDDLMLFGYHDTDSVTVKSFYNHEYYQFEKLEFADRSITRDELGKQGMALFGTDGDDDINDWGRNSVIDAGAGNDTINGSYGDDTLIGGTGNDILKGSYGADTYLFSKGHGQDVIYEYSDSANSKRDIDTLKFTDVNYAEVKFRRVGDDLMLFGYHDTDSVTVKSFYDHEYYQFEKLEFADRSISRDELIKAGLHLYGTDGNDEINDHADWDSILEGGKGNDILRGSYGADTYIFSKGHGQDVIYEYSDSANSKRDIDTLKFTDVNYAEVKFRRVDDDLMLFGYHDTDSVTVKSFYDHEYYQFEKLEFADRSITRDELGKQGMALFGTDGDDDINDWGRNSVIDAGAGNDTINGGYGDDTLIGGKGNDILKGSYGADTYIFSKGHGQDIVYEDTNNDNRARDIDTLKFTDVNYAEVKFRRVDNDLMLFGYHDTDSVTVKSFYSHVDYQFDKLEFADRSITRDELIKAGLHLYGTDGNDDIKDHADWDSILEGGKGNDILRGGYGADTYIFSKGHGQDIVYEDTNNDNRARDIDTLKFTDVNYAEVKFRRVDNDLMLFGYHDTDSVTIKSFYNHVDYQFDKLDFADRSITRDELGKQGMALFGTDGDDNINDWGRNSVIDAGAGNDTVNGGNGDDTLIGGKGNDILRGGYGADTYIFSKGHGQDIVYEDTNNDNRARDIDTLKFTDINLSELWFSRENNDLIIKSLLSEDKVTVQNWYSHQDHKIENIRLSNEQTLVSTQVEKMVESMAGFAQKHGGEISLASPEEVKQYINSLTAAL

SEQ ID NO: 39 - protein, 16 aa, Actinobacillus pleuropneumoniae
GGPLAYSNSPNSIPNA

SEQ ID NO: 40 - protein, 14 aa, Actinobacillus pleuropneumoniae
GAGGPLAYSNSPNS

SEQ ID NO: 41 - protein, 20 aa, Actinobacillus pleuropneumoniae
LVGAGGPLAYSNSPNSIPNA

SEQ ID NO: 42 - protein, 6 aa, Actinobacillus pleuropneumoniae
GSNRKD

SEQ ID NO: 43 - protein, 47 aa, Actinobacillus pleuropneumoniae
GAKGDDEIYGNDGHDILYGDDGNDVIHGGDGNDHLVGGNGNDRLIGG

SEQ ID NO: 44 - protein, 13 aa, Actinobacillus pleuropneumoniae
NNFLNGGDGDDEL

SEQ ID NO: 45 - protein, 20 aa, Actinobacillus pleuropneumoniae
GSRFTDIFHGAKGDDEIYGN

SEQ ID NO: 46 - protein, 20 aa, Actinobacillus pleuropneumoniae
DVIHGGDGNDHLVGGNGNDR

SEQ ID NO: 47 - protein, 20 aa, Actinobacillus pleuropneumoniae
IGGKGNNFLNGGDGDDELQV

SEQ ID NO: 48 - protein, 14 aa, Actinobacillus pleuropneumoniae
HGAKGDDEIYGNDG

SEQ ID NO: 49 - protein, 14 aa, Actinobacillus pleuropneumoniae
GGKGNNFLNGGDGD

SEQ ID NO: 50 - protein, 20 aa, Actinobacillus pleuropneumoniae
DGHDILYGDDGNDVIHGGDG

SEQ ID NO: 51 - protein, 20 aa, Actinobacillus pleuropneumoniae
RLIGGKGNNFLNGGDGDDEL

SEQ ID NO: 52 - protein, 15 aa, Actinobacillus pleuropneumoniae
GTGNDIISGGKDNDI

SEQ ID NO: 53 - protein, 19 aa, Actinobacillus pleuropneumoniae
HKTGDGNDSITDSGGQDKL

SEQ ID NO: 54 - protein, 20 aa, Actinobacillus pleuropneumoniae
FGGAGDDVIDGGNGNNFLVG

SEQ ID NO: 55 - protein, 20 aa, Actinobacillus pleuropneumoniae
YVHKTGDGNDSITDSGGQDK

SEQ ID NO: 56 - protein, 14 aa, Actinobacillus pleuropneumoniae
GGAGDDVIDGGNGN

SEQ ID NO: 57 - protein, 14 aa, Actinobacillus pleuropneumoniae
GGTGNDIISGGKDN

SEQ ID NO: 58 - protein, 14 aa, Actinobacillus pleuropneumoniae
KTGDGNDSITDSGG

SEQ ID NO: 59 - protein, 20 aa, Actinobacillus pleuropneumoniae
AGDDVIDGGNGNNFLVGGTG

SEQ ID NO: 60 - protein, 20 aa, Actinobacillus pleuropneumoniae
DIYVHKTGDGNDSITDSGGQ

SEQ ID NO: 61 - protein, 17 aa, Actinobacillus pleuropneumoniae
LTPGEENRERIQEGKNS

SEQ ID NO: 62 - protein, 12 aa, Actinobacillus pleuropneumoniae
WTVTDGDASSSV

SEQ ID NO: 63 - protein, 20 aa, Actinobacillus pleuropneumoniae
TPGEENRERIQEGKNSYITK

SEQ ID NO: 64 - protein, 14 aa, Actinobacillus pleuropneumoniae
LFRTPLLTPGEENR

SEQ ID NO: 65 - protein, 12 aa, Actinobacillus pleuropneumoniae
QGYDSGQGNGVQ

SEQ ID NO: 66 - protein, 60 aa, Actinobacillus pleuropneumoniae
VIDAGAGNDTVNGGNGDDTLIGGKGNDILRGGYGADTYIFSKGHGQDIVYEDTNNDNRAR

SEQ ID NO: 67 - protein, 14 aa, Actinobacillus pleuropneumoniae
NREEKIEYRREDDR

SEQ ID NO: 68 - protein, 20 aa, Actinobacillus pleuropneumoniae
PTNVGNREEKIEYRREDDRF

SEQ ID NO: 69 - protein, 62 aa, Actinobacillus pleuropneumoniae
GADGDDLLNGNDGDDILYGDKGNDELRGDNGNDQLYGGEGNDKLLGGNGNNYLSGGDGNDEL

SEQ ID NO: 70 - protein, 33 aa, Actinobacillus pleuropneumoniae
AGKGDDKLYGSSGSDLLDGGEGNDYLEGGDGSD

SEQ ID NO: 71 - protein, 16 aa, Actinobacillus pleuropneumoniae
STSGNHTIYDQGKSSD

SEQ ID NO: 72 - protein, 20 aa, Actinobacillus pleuropneumoniae
GDKGNDELRGDNGNDQLYGG

SEQ ID NO: 73 - protein, 20 aa, Actinobacillus pleuropneumoniae
LLGGNGNNYLSGGDGNDELQ

SEQ ID NO: 74 - protein, 14 aa, Actinobacillus pleuropneumoniae
GDKGNDELRGDNGN

SEQ ID NO: 75 - protein, 14 aa, Actinobacillus pleuropneumoniae
GGNGNNYLSGGDGN

SEQ ID NO: 76 - protein, 20 aa, Actinobacillus pleuropneumoniae
DGDDILYGDKGNDELRGDNG

SEQ ID NO: 77 - protein, 20 aa, Actinobacillus pleuropneumoniae
LLGGNGNNYLSGGDGNDELQ

SEQ ID NO: 78 - protein, 20 aa, Actinobacillus pleuropneumoniae
NGFNVLRAGKGDDKLYGSSG

SEQ ID NO: 79 - protein, 14 aa, Actinobacillus pleuropneumoniae
GITGKGDKLSSGKA

SEQ ID NO: 80 - protein, 9 aa, Actinobacillus pleuropneumoniae
LLEKKPDDF

SEQ ID NO: 81 - protein, 15 aa, Actinobacillus pleuropneumoniae
FDPTKGEIDISNSQT

SEQ ID NO: 82 - protein, 20 aa, Actinobacillus pleuropneumoniae
GITGKGDKLSSGKAYVDYFQ

SEQ ID NO: 83 - protein, 20 aa, Actinobacillus pleuropneumoniae
VFDPTKGEIDISNSQTSTLL

SEQ ID NO: 84 - protein, 14 aa, Actinobacillus pleuropneumoniae
KGDKLSSGKAYVDY

SEQ ID NO: 85 - protein, 14 aa, Actinobacillus pleuropneumoniae
EKKPDDFSKVVFDP

SEQ ID NO: 86 - protein, 14 aa, Actinobacillus pleuropneumoniae
FVTPLLTPGTESRE

SEQ ID NO: 87 - protein, 20 aa, Actinobacillus pleuropneumoniae
ELAGITGKGDKLSSGKAYVD

SEQ ID NO: 88 - protein, 20 aa, Actinobacillus pleuropneumoniae
TLLKFVTPLLTPGTESRERT

SEQ ID NO: 89 - protein, 84 aa, Actinobacillus pleuropneumoniae
EGKDTGFYGHAFYIERKNGGGSKNNSSGAGNSKDWGGNGHGNHRNNASDLNKPDGNNGNNQNNGSNQDNNSDVNAPNNPGRNYD

SEQ ID NO: 90 - protein, 66 aa, Actinobacillus pleuropneumoniae
VIDAGAGNDTINGGYGDDTLIGGKGNDILKGSYGADTYIFSKGHGQDIVYEDTNNDNRARDIDTLK

SEQ ID NO: 91 - protein, 440 aa, Artificial Sequence
  Recombinant ApxIV toxin
VIDAGAGNDTVNGGNGDDTLIGGKGNDILRGGYGADTYIFSKGHGQDIVYEDTNNDNRARGSTAEGKDTGFYGHAFYIERKNGGGSKNNSSGAGNSKDWGGNGHGNHRNNASDLNKPDGNNGNNQNNGSNQDNNSDVNAPNNPGRNYDGSTAGSTAVIDAGAGNDTINGGYGDDTLIGGKGNDILKGSYGADTYIFSKGHGQDIVYEDTNNDNRARDIDTLKGSTAGSTAFLNKLLSTAKERNRWEEPGQKLYNVEATSYAGSTAFLNKLLSTAKERNRWEEPGQKLYNVEATSYAGSTAFLNKLLSTAKERNRWEEPGQKLYNVEATSYAGSTAFLNKLLSTAKERNRWEEPGQKLYNVEATSYAGSTAFLNKLLSTAKERNRWEEPGQKLYNVEATSYAGSTAFLNKLLSTAKERNRWEEPGQKLYNVEATSYAGSTA

SEQ ID NO: 92 - protein, 442 aa, Artificial Sequence
  Recombinant ApxIV toxin with a HindIII site
VIDAGAGNDTVNGGNGDDTLIGGKGNDILRGGYGADTYIFSKGHGQDIVYEDTNNDNRARGSTAEGKDTGFYGHAFYIERKNGGGSKNNSSGAGNSKDWGGNGHGNHRNNASDLNKPDGNNGNNQNNGSNQDNNSDVNAPNNPGRNYDGSTAGSTAVIDAGAGNDTINGGYGDDTLIGGKGNDILKGSYGADTYIFSKGHGQDIVYEDTNNDNRARDIDTLKGSTAKLGSTAFLNKLLSTAKERNRWEEPGQKLYNVEATSYAGSTAFLNKLLSTAKERNRWEEPGQKLYNVEATSYAGSTAFLNKLLSTAKERNRWEEPGQKLYNVEATSYAGSTAFLNKLLSTAKERNRWEEPGQKLYNVEATSYAGSTAFLNKLLSTAKERNRWEEPGQKLYNVEATSYAGSTAFLNKLLSTAKERNRWEEPGQKLYNVEATSYAGSTA

SEQ ID NO: 93 - protein, 30 aa, Actinobacillus pleuropneumoniae
VIDAGAGNDTVNGGNGDDTLIGGKGNDILR

SEQ ID NO: 94 - protein, 4 aa, Actinobacillus pleuropneumoniae
GYGA

SEQ ID NO: 95 - protein, 20 aa, Actinobacillus pleuropneumoniae
AGAGNDTVNGGNGDDTLIGG

SEQ ID NO: 96 - protein, 20 aa, Actinobacillus pleuropneumoniae
SKGHGQDIVYEDTNNDNRAR

SEQ ID NO: 97 - protein, 14 aa, Actinobacillus pleuropneumoniae
DAGAGNDTVNGGNG

SEQ ID NO: 98 - protein, 14 aa, Actinobacillus pleuropneumoniae
DIVYEDTNNDNRAR

SEQ ID NO: 99 - protein, 20 aa, Actinobacillus pleuropneumoniae
SKGHGQDIVYEDTNNDNRAR

SEQ ID NO: 100 - protein, 20 aa, Actinobacillus pleuropneumoniae
KNGGGSKNNSSGAGNSKDWG

SEQ ID NO: 101 - protein, 20 aa, Actinobacillus pleuropneumoniae
HRNNASDLNKPDGNNGNNQN

SEQ ID NO: 102 - protein, 20 aa, Actinobacillus pleuropneumoniae
HRNNASDLNKPDGNNGNNQN

SEQ ID NO: 103 - protein, 14 aa, Actinobacillus pleuropneumoniae
ERKNGGGSKNNSSG

SEQ ID NO: 104 - protein, 14 aa, Actinobacillus pleuropneumoniae
GNSKDWGGNGHGNH

SEQ ID NO: 105 - protein, 14 aa, Actinobacillus pleuropneumoniae
KPDGNNGNNQNNGS

SEQ ID NO: 106 - protein, 14 aa, Actinobacillus pleuropneumoniae
DNNSDVNAPNNPGR

SEQ ID NO: 107 - protein, 20 aa, Actinobacillus pleuropneumoniae
NGGGSKNNSSGAGNSKDWGG

SEQ ID NO: 108 - protein, 20 aa, Actinobacillus pleuropneumoniae
NQNNGSNQDNNSDVNAPNNP

SEQ ID NO: 109 - protein, 30 aa, Actinobacillus pleuropneumoniae
VIDAGAGNDTINGGYGDDTLIGGKGNDILK

SEQ ID NO: 110 - protein, 4 aa, Actinobacillus pleuropneumoniae
SYGA

SEQ ID NO: 111 - protein, 19 aa, Actinobacillus pleuropneumoniae
GHGQDIVYEDTNNDNRARD

SEQ ID NO: 112 - protein, 20 aa, Actinobacillus pleuropneumoniae
DAGAGNDTINGGYGDDTLIG

SEQ ID NO: 113 - protein, 20 aa, Actinobacillus pleuropneumoniae
SKGHGQDIVYEDTNNDNRAR

SEQ ID NO: 114 - protein, 14 aa, Actinobacillus pleuropneumoniae
DAGAGNDTINGGYG

SEQ ID NO: 115 - protein, 14 aa, Actinobacillus pleuropneumoniae
DIVYEDTNNDNRAR

SEQ ID NO: 116 - protein, 20 aa, Actinobacillus pleuropneumoniae
NGGYGDDTLIGGKGNDILKG

SEQ ID NO: 117 - protein, 20 aa, Actinobacillus pleuropneumoniae
SKGHGQDIVYEDTNNDNRAR